(19) United States
(12) Patent Application Publication
(10) Pub. No.: US 2010/0129344 A1
(43) Pub. Date: May 27, 2010

(54) MEDICAL USES AND THERAPIES BASED UPON THE ACTION OF AZUROCIDIN ON IGFBP-1

(76) Inventors: Moira Sally Lewitt, Akersberga (SE); Kerstin Elisabet Hall, Solna (SE); Hans Jörnvall, Stockholm (SE)

Correspondence Address: BROWDY AND NEIMARK, P.L.L.C.;624 NINTH STREET, NW, SUITE 300, WASHINGTON, DC, 20001-5303, US

(21) Appl. No.: 12/091,004

(22) PCT Filed: Oct. 19, 2006
(86) PCT No.: PCT/GB2006/003885

§ 371 Date: Oct. 22, 2008

(30) Foreign Application Priority Data
Oct. 19, 2005 (GB) 0521276.6

(51) Int. Cl. A61K 39/395; A61K 38/16; A61P 13/12; A61K 38/08; C07K 7/06; A61K 38/10; A61K 38/02; A61P 3/00

(52) U.S. Cl. 424/130.1; 514/12; 514/2; 514/18; 514/17; 514/16; 514/15; 514/14; 514/13; 530/328

(57) ABSTRACT

The present invention concerns the use of azurocidin in the manufacture of a medicament for treating conditions associated with hepatic insulin resistance. Further provided is the use of a modulator of the interaction between azurocidin and IGFBP-1 in the manufacture of a medicament for use in the treatment of a condition which is associated with IGFBP-1 and/or IGF level and/or activity. Methods of treatment, an octopeptide modulator of amino acid sequence WDAISTYD, pharmaceutical compositions comprising the modulators of azurocidin and IGFBP-1 and uses thereof in the treatment of conditions associated with IGFBP-1 and/or IGF level and/or activity are also provided.

The present invention relates generally to medical uses and therapies based upon specific targeting of IGFBP-1 in a variety of conditions. These uses and therapies are predicated on the novel and surprising finding that azurocidin has potent IGFBP-1 specific protease activity, namely the identification, for the first time, of a specific interaction between IGFBP-1 and azurocidin. Thus, the present invention concerns the use of azurocidin in the manufacture of a medicament to treat conditions associated with hepatic insulin resistance and other conditions associated with elevated IGFBP-1 levels, specifically renal disease and diabetes-associated growth impairment in children. The invention also encompasses the modulation of the interaction between azurocidin and IGFBP-1 wherein said interaction can be enhanced or blocked. Hence, the invention encompasses modulators of the interaction between azurocidin and IGFBP-1 for use in therapy.

IGF-binding proteins (IGFBPs) are a family of six homologous proteins which have multifunctional roles through interactions with insulin-like growth factors (IGF-I and IGF-II) and other proteins in the matrix and on the cell surface. IGFBPs have been described as substrates for proteases which can modify the activity of IGFBPs, while post-translational modifications, for example phosphorylation can protect IGFBPs against proteolysis. IGFBPs have been proposed as regulators of insulin-like growth factors (small growth factors highly homologous to insulin, which can act to reduce glucose levels). These growth factors also function as paracrine/autocrine and endocrine hormones which stimulate proliferation and differentiation of a variety of cell types. To exert their effects, insulin-like growth factors interact with cell surface receptors, mainly with IGF receptor. IGFBPs can therefore act to bind IGFs and sequester them from the IGF receptor, to preclude IGF signalling. IGFBPs may therefore reduce or inhibit IGF action (at its receptor) and may thus “control” or regulate IGF action.

IGFBP-1 was first identified as an IGF-binding protein in human amniotic fluid. It is regarded as a glucose counter-regulator, increasing after insulin-induced hypoglycaemia and blocking the availability of IGF for stimulation of glucose uptake into the tissues. Hepatic IGFBP-1 is transcriptionally inhibited by insulin and stimulated by cytokines, glucocorticoids and cAMP- and AMPK-dependent pathways.

IGFBP-1 purified from amniotic fluid is a 28 kDa protein when run non-reduced on SDS-PAGE and has an association constant of approximately 6.6×10⁹ L/mol for IGF-I and 3.2×10⁹ L/mol for IGF-II. It is secreted from liver as a phosphoprotein and subsequent dephosphorylation reduces its affinity for IGF-I six fold. The circulating form of IGFBP-1, derived from liver is reported to be a single highly phosphorylated species, while during pregnancy, lesser phosphorylated forms of IGFBP-1 appear. The highly phosphorylated form of IGFBP-1 has a several fold higher affinity for IGF-I than non-phosphorylated IGFBP-1 but a similar affinity for IGF-II.

IGFBPs are substrates for kallikreins, neutral and acid-activated cathepsins and matrix metalloproteinases. Coppock et al. (Biology of Reproduction, 71, 438-443, 2004) reported that MMP-3 and MMP-9 can cleave IGFBP-1 and produce a series of fragments which are unable to bind IGF-1. It has further been reported that the phosphorylated form of IGFBP-1 is resistant to the action of such proteases (Gibson et al., Mol Hum Reprod., 7, 79-87, 2001). It has been proposed that IGFBP-1 fragments produced as a result of proteolytic cleavage may potentiate the effect of IGF on cell proliferation (Manes et al., 1997, J. Biol. Chem., 272, 25706-25712). Thus, proteolytic degradation may “convert” IGFBP-1 into forms which have opposite effects (as regards IGF action) to the intact IGFBP-1.

N-terminal fragments identified during purification of IGFBP-1 have been shown to be biologically active and able to bind IGF-I. However, whether these fragments are present in vivo, or were generated during the purification process, is not clear. Deletion of the 60 N-terminal amino acid residues of IGFBP-1 abolishes IGF-binding and mutations in the C-terminal region results in dimerisation and loss of IGF-binding. A loss of effect on IGF-I action is also seen when IGFBP-1, particularly in its dephosphorylated form, is polymerised by tissue transglutaminase. These multimeric forms of IGFBP-1 may in fact potentiate IGF action. It will be seen, therefore, that the way IGFBP-1 may “act” on IGF is complex, with both intact IGFBP-1 and other forms, notably fragments and multimeric forms, having effects, which may be opposing effects.

IGFBP-1 has an Arg-Gly-Asp (RGD) sequence at its C-terminus, through which it binds to the α₅ β₁ integrin receptor and may thus stimulate cell migration and induce apoptosis (Jones et al., PNAS, 90, 10553-10557, 1993; Perks et al., J. Mol. Endocrinol., 22, 141-150, 1999). This interaction of IGFBP-1 with its integrin receptor, in combination with activation of the IGF receptors by IGF-I, is required for the stimulation of wound healing by the combination of IGF-I and IGFBP-1.

Thus, in vitro, IGFBP-1 may be either an inhibitor of IGF when in phosphorylated form or a stimulator of IGF action (stimulation being mediated by other forms of IGFBP-1). Studies in vivo, however using a molar excess of IGFBP-1, generally result in reduced somatic growth and reduced glucose uptake.

Several conditions have been shown to be associated with an elevated level of IGFBP-1. Many of these conditions are associated with muscle wasting, where reduced availability of IGF-I for anabolism in tissues may be the consequence of this increase of IGFBP-1. In humans, high levels of IGFBP-1 are found for example after major surgery, in critical illness, type 1 diabetes mellitus, growth hormone deficiency, AIDS, aging, chronic renal failure, chronic liver disease and hyperthyroidism. As mentioned above, insulin normally regulates IGFBP-1 levels, and causes a reduction in IGFBP-1 expression. However, in some of these conditions insulin may have a reduced effectiveness in lowering IGFBP-1 concentrations and IGFBP-1 may be a marker for poor survival. In such patients where insulin has reduced effectiveness, it cannot be used as a means of lowering IGFBP-1, and hence an alternative therapy to insulin administration is required to lower IGFBP-1 concentrations in patients with elevated levels.

The present inventors have now identified for the first time a protease which acts to specifically cleave IGFBP-1 and hence reduce its levels, namely levels of the intact, inhibitory form of IGFBP-1. It is proposed that such a protease may be used therapeutically as a means of reducing levels of IGFBP-1 which are pathologically elevated. Previously, no IGFBP-1 specific proteases had been described and hence there were previously no known suitable alternatives to insulin, which could act to decrease elevated IGFBP-1 levels.

This novel protease is in fact a known protein, namely azurocidin (alternatively known in the art as cationic antimicrobial protein 37 (CAP37) or heparin-binding protein (HBP)). Azurocidin was originally identified from granules of human polymorphonuclear leukocytes (PMNs). The cDNA for azurocidin encodes 251 amino acids consisting of a mature protein of 225 residues, a signal sequence of 19 residues and a 7 amino acid propeptide. The three dimensional structure of azurocidin has been elucidated. There are 8 conserved cysteine residues that form 4 putative di-sulphide bridges, where the overall structure is homologous to neutrophil elastase. Azurocidin has been shown to have anti-microbial activity (“antibiotic” activity) and heparin binding activity. Azurocidin has further been shown to act as a multifunctional inflammatory mediator, which can attract monocytes to inflammation sites. It thus has potent chemoattractant or chemotactic properties. Such properties underlie the therapeutic utilities which have been proposed for azurocidin in treating infections and in promoting wound healing (see e.g. WO 03/092718, U.S. Pat. No. 5,837,247, WO 93/19087, WO 91/00907, U.S. Pat. No. 6,107,460, U.S. Pat. No. 6,071,879 and US 2004 048792).

However, although azurocidin is a member of the Serprocidin family of serine protease homologues, azurocidin was previously thought to be inactive as a protease. This was attributed to mutation of two members of its catalytic triad (Watorek, Acta Biochimica Polonica, 50, 743-752, 2003). It is therefore particularly surprising that the present inventors have now identified azurocidin as a specific protease for IGFBP-1. Based on this surprising finding it is proposed that azurocidin be used to reduce elevated levels of IGFBP-1 and treat conditions associated therewith. In this regard, the present inventors propose that azurocidin may be used to treat conditions associated with hepatic insulin resistance.

In a first embodiment, the present invention thus provides use of azurocidin in the manufacture of a medicament for treating conditions associated with hepatic insulin resistance.

The term “azurocidin” as used herein includes all known forms of the azurocidin protein, as identified also by the terms “CAP37” (cationic anti-microbial protein 37) and “HBP” (Heparin-Binding Protein). Azurocidin has been described extensively in the literature, including also in the patent specifications mentioned above. Further mention may be made also of WO 03/076459, U.S. Pat. No. 5,877,151, WO 99/26647 and EP 0645016 in which descriptions of azurocidin/HBP/CAP37 are provided. (Insofar as all the listed patent specifications refer to azurocidin/HBP/CAP37 proteins and fragments or derivatives or analogues thereof, they are incorporated herein by reference.) Fragments of azurocidin or derivatives or analogues thereof may retain azurocidin activity and are included herein. In this respect, it will particularly be noted that the fragments, derivatives or analogues etc. retain the activity of azurocidin in cleaving IGFBP-1.

As noted above, the sequence of the DNA encoding human azurocidin has been elucidated and is publicly available, as is the translated amino acid sequence. Other “azurocidin” sequences have also been published.

Azurocidin proteins have been described in a number of species, but preferably the azurocidin may be of mammalian, more preferably human origin.

In particular, the term “azurocidin” as used herein relates to azurocidin having an amino acid sequence as shown in SEQ ID NO. 1 or a functionally equivalent variant, derivative or fragment thereof. The amino acid sequence of SEQ ID NO: 1 relates to the fully processed azurocidin protein i.e. without the N-terminal pre and prepro sequences. The processing of azurocidin is well documented in the art and the difference between the initial translated sequence and the final processed sequence reported. Hence, “azurocidin” as used herein also relates to the unprocessed azurocidin sequence (SEQ ID NO. 4) and to the pre-azurocidin sequence (SEQ ID NO. 2) and the preproazurocidin sequence (SEQ ID NO. 3) and to functionally equivalent variants, derivatives and fragments thereof. Alternatively viewed the azurocidin may be encoded by a polynucleotide sequence as shown in SEQ ID NO. 5, or a functionally equivalent variant or fragment thereof.

Variants of azurocidin may include, for example, different allelic variants as they appear in nature e.g. in other species or due to geographical variation etc. Functionally equivalent variants may also include polypeptides which incorporate one or more amino acid substitutions, or intrasequence or terminal deletions or additions to the above sequence. Functionally equivalent derivatives may include chemical modifications of the amino acid sequence, including for example the inclusion of chemically substituted or modified amino acid residues. All such variants and derivatives are included provided they retain the ability to treat conditions associated with hepatic insulin resistance and/or act as a protease for IGFBP-1. More particularly, the variants, derivatives and fragments are functionally equivalent in that they exhibit at least 5%, 10%, 20%, 30% or 40%, preferably at least 50% or 60% or 70% or 80% of the protease activity of azurocidin of SEQ ID NO. 1, 2, 3 or 4. Protease activity may be measured using a synthetic fragment of IGFBP-1 attached to a chromagenic substrate or using methods as described in the Examples; visualising IGFBP-1 fragments after SDS-PAGE (by labelling IGFBP-1 or silver staining). Also included within the scope of the invention is the use of “non-native” isomers of “native” L-amino acid azurocidin sequences e.g. peptides containing D-amino acid isomers.

It is known in the art to modify the sequences of proteins or peptides, whilst retaining their useful activity and this may be achieved using techniques which are standard in the art e.g. random or site directed mutagenesis, cleavage and ligation of nucleic acids, chemical peptides synthesis etc.

Preferably, amino acid changes are of a minor nature, that is conservative amino acid substitutions that do not significantly affect the folding and/or activity of the protein; small deletions, typically of 1 to 30 amino acids; small amino- or carboxyl-terminal extensions; addition of a small linker peptide of up to about 20-25 residues; or a small extension that facilitates purification by changing net charge or another function, such as a poly-histidine tract, an antigenic epitope or a binding domain. Hence, N and/or C extensions to the protein or peptides are included in the definition. The lengths of each extended derivative may vary, for example, derivatives may be extended by up to 50, 30, 20, 10 or 5 amino acids. Examples of conservative substitutions are within the group of basic amino acids (such as arginine, lysine and histidine), acidic amino acids (such as glutamine and asparagine), hydrophobic amino acids (such as leucine, isoleucine and valine), aromatic amino acids (such as phenylalanine, tryptophan and tyrosine) and small amino acids (such as glycine, alanine, threonine and methionine).

The azurocidin preferably exhibits at least 50%, 60%, 70%, 80% or 90% sequence identity or similarity to the amino acid sequence of SEQ ID NO. 1, 2, 3 or 4. More preferably the azurocidin exhibits at least 95, 97, 98 or 99% identity or similarity to the sequence of SEQ ID NO. 1, 2, 3 or 4.

Alternatively viewed, azurocidin can be encoded by the nucleotide sequence shown in SEQ ID NO. 5 or a nucleotide sequence having at least 50%, 60%, 70%, 80% or 90% identity thereto. Preferably, the azurocidin is encoded by a nucleic acid sequence with at least 95, 97, 98 or 99% identity to SEQ ID NO. 5. The nucleic acid sequence may be of genomic, cDNA, RNA or synthetic origin or any combination thereof. The degree of identity between two nucleic acid and two amino acid sequences may be determined by means of computer programs known in the art such as GAP provided in the GCG program package (Needleman and Wunsch, 1970, Journal of Molecular Biology 48: 443-453). For the purposes of determining the degree of identity between two nucleic acid sequences for the present invention, GAP may be used with the following settings: GAP creation penalty of 5.0 and GAP extension penalty of 0.3. For the purposes of determining the degree of identity between 2 amino acid sequences, GAP can be used with the following settings: GAP creation penalty of 3.0 and GAP extension penalty of 0.1. Amino acid similarity may be measured using the Best Fit program of GCG Version 10 Software package from the University of Wisconsin. This program uses the local homology algorithm of Smith and Waterman with the default values: Gap creation penalty=8, Gap extension penalty=2, Average match=2.912, Average mismatch=2.003.

The azurocidin may also be encoded by a nucleic acid sequence that hybridises to a nucleic acid sequence of SEQ ID NO. 5 under high stringency conditions defined herein as: prehybridisation and hybridisation at 42° C. in 5×SSPE, 0.3% SDS, 200 μg/ml sheared and denatured salmon speron DNA and 50% formamide. The carrier material is washed three times for 30 minutes using 2×SSC, 0.2% SDS at least 70° C.

Azurocidin used in the present invention may be prepared synthetically by established techniques or by recombinant technology. Hence, azurocidin may be produced recombinantly from its encoding nucleic acid which can also be produced synthetically e.g. in an automatic DNA synthesizer or may be isolated and cloned from genomic DNA. The nucleic acid can be inserted into a recombinant expression vector, e.g. a plasmid, where the nucleic acid encoding azurocidin may be operably connected to a suitable promoter to allow expression in a particular cell. Techniques and materials for recombinant expression are well known, and any desirable or convenient vector may be used. The vector may for example be a plasmid, bacteriophage, or cosmid into which a nucleic acid (encoding the azurocidin) may be inserted or cloned. Such vectors preferably contain one or more unique restriction sites and may be capable of autonomous replication in a defined host cell including a target cell or tissue or may be integrable with the genome of the defined host such that the cloned sequence is reproducible. The choice of the vector will depend on the compatibility of the vector with the host cell into which the vector is to be introduced. The vector may also include a selection marker and regulatory elements to control expression of the inserted nucleic acid. Examples of suitable promoters for transcribing the nucleic acid sequence in mammalian cells include the SV40 promoter, the MT-I promoter, Rous Sarcoma virus promoter, cytomegalovirus promoter and a bovine papilloma virus promoter. Suitable promoters for expression in bacteria include for example the promote obtained from the E. coli lac operon.

Hence, expression of azurocidin may occur from its encoding nucleic acid e.g. from a vector containing the nucleic acid in a host cell. Such a cell may be prokaryotic or eukaryotic and may be mammalian, insect, bacterial or fungal. Azurocidin produced recombinantly may be purified by heparin affinity chromatography with a salt gradient for elution or by size exclusion chromatography e.g. using a Bio-Sil TSK-125 size exclusion column (Bio-Rad Laboratories) and eluted using an HPLC system.

Alternatively, azurocidin may be isolated from azurophil granules of PMN, preferably from azurophil granules of human PMN. Azurocidin may also be isolated from other biological tissues or samples, for example from urine. Isolation of azurocidin from PMN can be carried out by isolating PMN from blood and lysing PMN by standard techniques e.g. by nitrogen cavitation. Azurophil granules can be separated on discontinuous Percoll density gradients. Azurophil granule membrane associated material can be obtained by carrying out freeze-thaw cycles in dry ice or acetone with sonication pulses between each cycle, centrifugation, extraction with 50 mM glycine, pH 2.0 and further centrifugation. Azurocidin can be purified using for example a Bio-Sil TSK-125 size exclusion column (Bio-Rad Laboratories) and eluted using an HPLC system. Conveniently, the heparin-binding properties of azurocidin may be utilised to enable its purification by heparin affinity chromatography, for example using heparin-agarose gel which is available from Pharmacia.

In the present invention azurocidin is used to treat “conditions associated with hepatic insulin resistance”. This term includes any condition or disease state which results from a reduced action of insulin on liver cells (hepatocytes). Such a reduction of insulin action may be a 30% reduction or more (e.g. a 40%, 50%, 60%, 70%, 80%, 90% or 100% reduction). The reduction can be measured directly using the isotope method or indirectly (liver fat) as described further below. In such conditions, the liver is accordingly less sensitive to, or less responsive to, the effects of insulin. The mechanism for this “resistance” is not entirely elucidated, but may be due (at least in part) to reduced effectiveness of insulin action or its receptors (including for example reduced binding of insulin to receptors, reduced receptor expression or reduced signalling via the receptor. Hepatic insulin resistance is associated at the cellular level with reduced phosphorylation of insulin receptors and phosphoinositol-3 kinase mediate protein kinase B, which may result in a reduced ability to suppress hepatic glucose output, VLDL triglyceride production, and also IGFBP-1 production. A clear manifestation of hepatic insulin resistance is increased hepatic glucose output (leading to increased fasting blood glucose). This may also contribute to post prandial hyperglycaemia. The increased VLDL triglyceride production which is seen may lead to hyperlipidaemia.

Hence, conditions associated with hepatic insulin resistance include conditions associated with a reduced ability to suppress hepatic glucose output, VLDL triglyceride production and also IGFBP-1 production. Such conditions may exhibit post prandial hyperglycaemia and hyperlipidaemia. These may be accompanied by visceral obesity, increased cardiovascular morbidity and mortality. Consequences of impaired IGFBP-1 suppression due to hepatic insulin resistance include inhibition of growth in childhood, inhibition of IGF-stimulated glucose uptake, and reduced feedback inhibition of growth hormone secretion.

An important feature of hepatic insulin resistance is, thus, that the effect of insulin in suppressing IGFBP-1 levels, which is normally seen, is decreased. Thus decreased suppressibility of IGFBP-1 by insulin (whether exogenous or endogenous (post-prandial)) characterises hepatic insulin resistance (at least in one aspect), and may lead to increased levels of IGFBP-1 (i.e. circulating levels of IGFBP-1 may be increased, leading to a decrease in IGF actions in target tissues). This may have serious consequences on IGF action in the body.

More particularly, the present invention is used to treat conditions associated with hepatic insulin resistance in which serum IGFBP-1 levels are elevated, more specifically, the IGFBP-1 levels are elevated in relation to insulin. IGFBP-1 levels are elevated as compared to subjects not suffering from hepatic insulin resistance, or from the condition in question. The IGFBP-1 levels may be seen to be inappropriately elevated, or elevated above normal, normal being defined as in a healthy subject. More particularly, elevated levels of IGFBP-1 are those that are 2 standard deviations or more above the mean for the assay used. The level must be judged in relation to a healthy control population using that assay. Furthermore the level may be judged in relation to the insulin levels.

In relation to insulin, an elevated IGFBP-1 level may be seen as a value that lies 2 standard deviations or more above the regression line between log IGFBP-1 and log insulin for a healthy control population.

Conditions which may be treated by azurocidin according to the invention thus include critical illness and intensive care, when associated with hepatic insulin resistance, type 2 diabetes mellitus and impaired glucose tolerance, and type 1 diabetes associated with hepatic insulin resistance. Azurocidin may be used according to the present invention to treat patients with hepatic insulin resistance in intensive care and/or who are critically ill. “Critical illness” and “intensive care” are recognised in the art as conditions in which patients undergo a range of potentially very serious metabolic changes and reactions, which are life-threatening, and which may ultimately lead to death. Clinicians use a scoring system for severity of illness, called the APACHE-II score, that takes into account a variety of parameters, including age, immune compromise and biochemical measurements. An APACHE-II score on admission of 10 or more may reflect patients in a critical condition who may be treated according to the present invention (Knaus et al., Critical Care Medicine, 13, 820, 1981).

In all cases, in the conditions which may be treated according to the present invention, they are further associated with hepatic insulin resistance.

Hepatic insulin resistance can be detected using stable isotopes of glucose to measure hepatic glucose production in response to insulin. An at least 30% reduction may be indicative of hepatic insulin resistance. Alternatively fat accumulation in the liver may be measured for example by magnetic resonance imaging (2 standard deviations or more above the mean for a healthy control population may be indicative of hepatic insulin resistance. This corresponds to approximately greater than 2% fat content in liver).

For administration azurocidin is conveniently formulated into a pharmaceutical composition. Such compositions may comprise azurocidin together with at least one pharmaceutically acceptable carrier, diluent or excipient. The active ingredients in such compositions may comprise from 0.05% to 99% by weight of the formulation.

By “pharmaceutically acceptable” is meant that the ingredients must be compatible with the other ingredients of the composition as well as physiologically acceptable to the recipient. Pharmaceutical compositions comprising azurocidin may be formulated according to techniques and procedures well known in the art. Other ingredients may also be included, for example stabilisers, preservatives etc. Formulations may be in the form of sterile aqueous solutions and/or suspensions of the pharmaceutically active ingredients, aerosols, ointments and the like.

Azurocidin and/or formulations thereof may be administered by any suitable method known in the medicinal arts including oral, parenteral, topical, subcutaneous administration or by inhalation. Preferably, azurocidin is administered parenterally or topically. Hence, administration may be carried out by cutaneous, subcutaneous, intraperitoneal or intravenous injection. Administration of azurocidin may be in a single dose to be taken at regular intervals or may be administered as divided doses to be taken for example during the course of a day. Alternatively, azurocidin may be administered as a sustained release formulation which may be given at longer intervals. The precise dosage of the active compound to be administered, the number of doses and the length of the course of treatment will depend on a number of factors, including age of the patient. However, preferably, a typical dose will result in tissue levels of azurocidin of 1-10 nmol/L i.e. 1, 2, 3, 4, 5, 6, 7, 8, 9 or 10 nmol/L. The dose of azurocidin to be administered can be from 1 to 200 nmol/L, for example 10, 20, 30, 40, 50, 60, 70, 80, 90, 100, 120, 140, 160 or 180 mmol/L.

The compositions which can be used in the present invention may be formulated according to techniques known in the art and may comprise any known carrier, diluent or excipient. For example, formulations which are suitable for parenteral administration conveniently comprise sterile aqueous solutions and/or suspensions of pharmaceutically active ingredients preferably made isotonic with the blood of the recipient, generally using sodium chloride, glycerin, glucose, mannitol, sorbitol and the like. When administered orally, the azurocidin may be in the form of a tablet, capsule, powder, solution or elixir. When administered in tablet form, the pharmaceutical composition may additionally contain a solid carrier such as a gelatin or an adjuvant. Generally speaking, for oral administration, the composition may need to be provided with a coating or in a form which provides protection from enteric degradation or digestion. The tablet, capsule or powder preferably contains from about 5 to 95% peptide. When administered in liquid form, a liquid carrier such as water, petroleum, oils of animal or plant origin such as peanut oil, mineral oil, soybean oil or sesame oil or synthetic oils may be added. The liquid form of the composition may also contain physiological saline solution, dextrose or other saccharide solution or glycols. When administered in liquid form the pharmaceutical composition preferably contains from about 0.5 to 90% by weight of peptide. Compositions of azurocidin suitable for topical administration may comprise azurocidin in sterile formulation mixed with known suitable ingredients such as paraffin, daserine, cetamol, glycerol and its like to form suitable ointments or creams.

Viewed in an alternative aspect, the present invention also provides a method of treating a condition associated with hepatic insulin resistance, said method comprising administering azurocidin to a subject in need thereof (a subject suffering from a condition associated with hepatic insulin resistance). This may be a patient responsive to such azurocidin therapy. Preferably the subject is a human.

It will be noted from the above that azurocidin may have a utility in treating any condition associated with elevated IGFBP-1 levels, and not only those associated with hepatic insulin resistance. Such conditions associated with elevated IGFBP-1 levels may benefit from reducing the IGFBP-1 level by cleavage of IGFBP-1 using azurocidin. Specifically, the present invention extends to the use of azurocidin in treating such conditions, which are renal disease which may be associated with an increased IGFBP-1 level and growth impairment in children with diabetes.

Accordingly, in a further aspect, the present invention also provides the use of azurocidin in the manufacture of a medicament for treating renal disease or for treating growth impairment in children with diabetes.

For such use the azurocidin may be formulated and administered as earlier described. Alternatively viewed, this aspect of the invention also provides a method of treating renal disease or growth impairment in children with diabetes, said method comprising administering azurocidin to a subject in need thereof.

It is a feature of the present invention that the dosage levels of azurocidin required for therapeutic efficacy (in cleaving IGFBP-1) are much lower than the dosages here before used in the art to treat other conditions, for example for the anti-microbial and wound healing effects of azurocidin.

Thus, the doses may be at least 1000 times less than the doses used for anti-microbial and wound healing effects or any other effect based on IGFBP-1's previously known IGF-dependent and -independent actions. The IGFBP-1-specific protease effect of azurocidin is unexpectedly very potent which means that much lower doses may be used in hepatic insulin resistance (and other conditions associated with elevated IGFBP-1) than would be expected to be the case. The advantage of using such lower doses is that other actions of azurocidin may be minimised.

As described previously, the rationale behind the invention is the identification of azurocidin as a protease for IGFBP-1. The inventors have identified for the first time a novel interaction between azurocidin and IGFBP-1. Both azurocidin and IGFBP-1 are important multi-functional molecules which occur in the body, and this interaction as it occurs in the body may have important physiological effects. It has been explained above that not only is IGFBP-1 functionally active and important in its own right, as a binder for IGF which may regulate the bioavailability of IGF, and hence its physiological effects, but also fragments of IGFBP-1, specifically proteolytic fragments thereof, may also have effects in promoting the action of IGF. Hence, proteolytic fragments may potentiate the effect of IGF on cell proliferation. Further, proteolytic fragments of IGFBP-1 may interact with IGF-1 with lower affinity than IGFBP-1. We have further interestingly observed, and report herein, that proteolytic fragments of IGFBP-1 may also directly stimulate glucose uptake, which may be an IGF-independent effect. Thus, the interaction between azurocidin and IGFBP-1 may be important in regulating the levels of IGFBP-1 and IGFBP-1 fragments respectively, which in turn regulate the levels and/or activity of IGF.

A number of different conditions may be associated with perturbed levels and/or activity of IGF or IGFBP-1. (This may be either causatively or symptomatically). We have described above a number of pathological conditions in which IGFBP-1 levels are elevated, most notably when associated with hepatic insulin resistance (although this is not a necessary corollary of elevated IGFBP-1). In such conditions, as proposed above, the degradation of IGFBP-1 may be useful, to forms of IGFBP-1 (fragments) which no longer block or inhibit IGF action, but which may promote IGF action. The present inventors have further interestingly observed that the cleavage products of azurocidin action on IGFBP-1 (i.e. the azurocidin proteolytic fragments) retain the known effect of IGFBP-1 on integrin receptors (leading to inter alia promotion of wound healing). Thus, azurocidin may degrade IGFBP-1 to forms which no longer inhibit or block IGF action, while preserving the effect of IGFBP-1 on integrin receptors.

As proposed above, one way of achieving this is to administer azurocidin. Alternatively, the effect of endogenous azurocidin in cleaving IGFBP-1 may be enhanced. As will be described in more detail further below, the inventors have surprisingly observed that aprotinin may enhance the action of azurocidin in cleaving IGFBP-1.

Conversely other conditions may be associated with excessive or unwanted IGF levels or action. There are also conditions where elevated or high IGF levels or indeed normal IGF levels are undesirable (for example such as cancer or atherosclerosis where the growth promoting effects of IGF are undesirable). In such conditions, it would be desirable to be able to reduce any increase in azurocidin which might occur (for example as a result of an inflammatory response in which azurocidin is released), in order to minimise or prevent an increase in free IGF levels and/or IGF activity resulting from azurocidin action. In such conditions, it would be desirable to block cleavage of IGFBP-1 by azurocidin, i.e. block the azurocidin-IGFBP-1 interaction.

This novel observation of IGFBP-1 specific azurocidin protease activity (i.e. of an interaction between IGFBP-1 and azurocidin) thus leads to a number of related applications, beyond administration of azurocidin. Specifically, such related applications involve modulation of the interaction between azurocidin and IGFBP-1. By enhancing the interaction, IGFBP-1 levels (namely levels of the intact, inhibitory form of IGFBP-1) may be reduced. By blocking the interaction, IGFBP-1 levels may be maintained or increased; the degradation of endogenous IGFBP-1 may be decreased, and its inhibitory effect on IGFs may be preserved. Modulators of the interaction may thus be used to treat any condition which is associated with IGFBP-1 and/or IGF levels or activity. These may be conditions associated with aberrant levels and/or activity of IGFBP-1 or IGF e.g. elevated levels of IGFBP-1 or elevated or excessive levels of IGF or unwanted or excessive or elevated IGF action.

Any condition which is responsive to, or which benefits from reduction of IGFBP-1 or from reduction in IGF levels or activity, by enhancing or blocking the interaction respectively, may be treated.

Accordingly, in a further aspect the present invention provides a modulator of the interaction between azurocidin and IGFBP-1 for use in therapy.

Such a modulator may be any agent which blocks (i.e. inhibits or reduces) or which enhances the interaction.

As noted above, such therapy may be the therapy of any condition which is associated with IGFBP-1 and/or IGF level and/or activity. These may be conditions associated with aberrant (e.g. elevated) or unwanted levels or activity.

Accordingly, alternatively viewed, this aspect of the invention also provides use of a modulator of the interaction between azurocidin and IGFBP-1 in the manufacture of a medicament for use in the treatment of a condition which is associated with IGFBP-1 and/or IGF level and/or activity.

In a further embodiment, this aspect of the invention also provides a method of modulating the interaction between azurocidin and IGFBP-1 said method comprising administering a modulator of such interaction to a subject in need thereof.

More particularly, this aspect of the invention provides a method of treating a condition which is associated with IGFBP-1 and/or IGF level and/or activity, said method comprising administering a modulator of the interaction between azurocidin and IGFBP-1 to a subject in need thereof.

The modulator may enhance, i.e. increase or improve, the interaction between azurocidin and IGFBP-1. Preferably, the interaction may be improved by at least 10%, 20%, 30%, 40%, 50%, 60%, 70%, 80% or 90%. By enhancing the interaction, cleavage of IGFBP-1 is enhanced. Alternatively, the modulator may block or inhibit the interaction between azurocidin and IGFBP-1. Hence, the interaction will be decreased or reduced by administration of the modulator. The interaction is preferably reduced by at least 10%, 20%, 30%, 40%, 50%, 60%, 70%, 80% or 90%. By blocking the interaction, cleavage of IGFBP-1 is reduced. It may be prevented.

The modulator can be administered to block or enhance the azurocidin and IGFBP-1 interaction which occurs naturally in a patient i.e. the interaction between endogenous azurocidin and endogenous IGFBP-1. Alternatively, the modulator can be administered to block or enhance the interaction which occurs as a result of the administration of exogenous azurocidin to a patient, for example azurocidin which has been administered to treat hepatic insulin resistance-associated conditions as described above, or any other condition associated with elevated IGFBP-1 levels. Hence, alternatively viewed, the interaction between exogenous azurocidin and endogenous IGFBP-1 may be modulated.

The modulator may be any agent, e.g. molecule or entity which has the desired activity (or effect) in modulating the interaction. The modulator may be a small organic molecule, or a more complex chemical structure, or a protein or polypeptide (including both short peptides or peptide fragments and longer polypeptide molecules), or a nucleic acid. It may be any chemical entity with the desired functional property of modulating the interaction.

Modulators of the interaction can be identified by using screening methods known in the art and extensively described in the literature. Thus, known or existing molecules may be screened for activity in modulating the interaction. Further, novel molecules may be synthesized and screened for such activity. A vast range of molecules and molecule libraries exist which could be screened, or libraries of molecules may be generated for screening, using methods known and described in the art for library generation, e.g. combinatorial libraries based on chemical synthesis. Computer software may be used to predict or design molecular structures, or to facilitate the design, based on known information pertaining to azurocidin and/or IGFBP-1 sequence and/or structure. Structure/function studies may be carried out to facilitate this process.

Libraries based on “biomolecules” e.g. peptide or protein molecules may also be generated using known methods and screened. For example phage display or other peptide-display libraries may be constructed and screened, or libraries based on antibody structures (e.g. affibodies). Aptamer libraries may also be generated and screened.

n-Hybrid screening assays are well known and described in the art for use in screening for inhibitors of a desired interaction. Such assays could also be used to screen for enhancers of a desired interaction. For example, yeast-2-hybrid and reverse yeast-2-hybrid assays can be used to identify modulators which enhance or block the azurocidin and IGFBP-1 interaction. An n-hybrid assay may be used to find a binding partner for azurocidin, that could then be applied in a protease assay.

Modulators of the interaction may also be identified by screening assays based on detecting modulation of the protease activity of azurocidin on IGFBP-1 (i.e. detecting whether or not enhancement or inhibition of the activity has taken place). For example an in vitro functional activity-based screening assay may be performed by adding a putative modulator (i.e. test agent or compound) to a reaction mixture comprising azurocidin and IGFBP-1 under conditions which enable the protease cleavage reaction to take place, and detecting whether or not the reaction is modulated (e.g. enhanced or inhibited) compared to the reaction in the absence of inhibitor. Protease activity may be detected and monitored or measured in any convenient way. For example, by using a synthetic fragment of IGFBP-1 (with isoleucine at P1) attached to a chromagenic substrates (e.g. succinyl-(X)ₙ-I-nitroaniline where X is any amino acid, and n is any integer e.g. 1 to 9) or using the methods described in the Examples below, visualising IGFBP-1 fragments after SDS-PAGE (by labelling IGFBP-1 or by silver staining).

In one embodiment, the modulator blocks (or inhibits) the interaction. Such a modulator may be viewed as an antagonist of the azurocidin and IGFBP-1 interaction. In a preferred embodiment the antagonist (or modulator which blocks or otherwise inhibits the interaction) is an antibody. The term “antibody” is used herein broadly to encompass any type of “antibody”-based molecule, including both native and synthetic or modified antibodies, as well as antibody fragments or derivatives of antibody molecules, with the proviso that any such antibody molecules, or fragment or derivative retains binding activity to the target molecule (e.g. azurocidin), and the ability to block the interaction. The term thus includes both monoclonal and polyclonal antibodies and these may be selected from naturally occurring antibodies or antibodies which have been specifically raised to the target antigen. Alternatively, fragments of antibodies may be used, such as Fab fragments or Fv fragments, or any other fragments retaining binding activity. Furthermore, the term “antibody” extends to recombinant and synthetic antibodies and to antibody hybrids. Such antibodies are now well known in the art and include for example chimeric antibodies, humanised antibodies, CDR-grafted antibodies, single-chain antibodies etc. A skilled practioner would readily be able to prepare an appropriate “antibody” of choice, using techniques well-described in the literature.

The antibody is preferably directed against azurocidin i.e. is an anti-azurocidin antibody. An anti-azurocidin antibody may be readily obtained, as described above. Anti-azurocidin antibodies are also publicly available, for example monoclonal anti-human azurocidin/CAP37/HBP antibody, catalogue number MAB2200 (Clone 246322) or polyclonal antibody catalogue number AF2200, goat anti-human azurocidin both of R&D systems.

The use of an anti-azurocidin antibody represents a preferred aspect of the present invention, and accordingly in this preferred aspect, the invention provides an anti-azurocidin antibody for use in therapy and a pharmaceutical composition comprising an anti-azurocidin antibody, together with at least one pharmaceutically acceptable carrier, diluent or excipient.

Alternatively, it may be an anti-IGFBP-1 antibody. IGFBP-1 antibodies are publically available, for example, the following monoclonal IGFBP-1 antibodies: MAb 6301, 6302, 6303, 6304, 6305 of MediX Biochemica, Finland; and DSL-R00334 and DSL-R00335 of Diagnostic Systems Laboratories (Webster, Tex., USA).

Alternatively an antagonist (or blocking modulator) may be any other molecule or entity which binds to the active site of azurocidin to prevent its activity, e.g. a blocking peptide which may be identified using techniques known in the art.

An antagonist (or blocking modulator) may also be a molecule or entity which acts as an alternative substrate for azurocidin i.e. an alternative substrate which may compete with endogenous IGFBP-1 and specifically reduce the action of azurocidin on IGFBP-1. The benefit of such a blocking modulator is that the other actions of azurocidin may be preserved. Such a “competitor” modulator may, for example, be a fragment of IGFBP-1, or a synthetic peptide, which contains the specific cleavage site in IGFBP-1 for azurocidin. The specific cleavage site, as determined by N- and C-terminal sequence analysis of HPLC-purified fragments (see Example 1), is at Ile¹³⁰-Ser¹³¹. Peptides comprising such a specific cleavage site (specifically comprising isoleucine e.g. I¹³⁰ or a corresponding or equivalent residue) may accordingly be used as such blocking modulators. The first proteolytic cleavage site of azurocidin in IGFBP-1 is Ile¹³⁰, and hence this Ile residue is accordingly recognised as P1. Such a peptide may, for example, be from 4 to 20 amino acids long and comprises an Ile residue (more particularly an Ile residue equivalent to or corresponding to I¹³¹, or, alternatively put, to Ile at P1) and is capable of acting as substrate for azurocidin protease activity. Preferably, such a peptide is from 4 to 15, 4 to 12, 4 to 10, 4 to 9 or 4 to 8 amino acids, or 5 to 20, 5 to 15, 5 to 12, 5 to 10, 5 to 9 or 5 to 8 amino acids more preferably 6 to 20, 6 to 15, 6 to 12, 6 to 10 or 6 to 8 amino acids.

An exemplary such peptide may for example be

(X)ₙ—I—(X)ₘ, (Formula I)

wherein X is any amino acid; n is 1 to 10, (e.g. 1 to 9, 1 to 6, 1 to 5 or 1 to 4; and m is 0 to 10 (e.g. 0 to 9, 0 to 6, 0 to 5, 0 to 4, 1 to 9, 1 to 6, 1 to 5 or 1 to 4). n and m may be the same or different.

X may be any amino acid, whether natural or synthetic, conventional or non-conventional. In addition to the well known 20 conventional amino acids (Ala(A); Cys(C); Asp(D); Glu(E); Phe(F); Gly(G); His(H); Ile(I); Lys(K); Leu(L); Met(M); Asn(N); Pro(P); Gln(O); Arg(R); Ser(S); Thr(T); Val(V); Trp(W); and Tyr(Y)), a number of non-conventional amino acids are shown in Table 1 below.

TABLE 1

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropanecarboxylate | Cpro | L-N-methylasparagine | Nmasn |
| aminoisobutyric acid | Aib | L-N-methylaspartic acid | Nmasp |
| aminonorbornylcarboxylate | Norb | L-N-methylcysteine | Nmcys |
| cyclohexylalanine | | L-N-methylglutamine | Nmgln |
| cyclopentylalanine | Cpen | L-N-methylglutamic acid | Nmglu |
| D-alanine | Dal | Chexa L-N-methylhistidine | Nmhis |
| D-arginine | Darg | L-N-methylisolleucine | Nmile |
| D-aspartic acid | Dasp | L-N-methylleucine | Nmleu |
| D-cysteine | Dcys | L-N-methyllysine | Nmlys |
| D-glutamine | DgIn | L-N-methylmethionine | Nmmet |
| D-glutamic acid | Dglu | L-N-methylnorleucine | Nmnle |
| D-histidine | Dhis | L-N-methylnorvaline | Nmnva |
| D-isoleucine | Dile | L-N-methylomithine | Nmorn |
| D-leucine | Dleu | L-N-methylphenylalanine | Nmphe |
| D-lysine | Dlys | L-N-methylproline | Nmpro |
| D-methionine | Dmet | L-N-methylserine | Nmser |
| D-ornithine | Dorn | L-N-methylthreonine | Nmthr |
| D-phenylalanine | Dphe | L-N-methyltryptophan | Nmtrp |
| D-proline | Dpro | L-N-methyltyrosine | Nmtyr |
| D-serine | Dser | L-N-methylvaline | Nmval |
| D-threonine | Dthr | L-N-methylethylglycine | Nmetg |
| D-tryptophan | Dtrp | L-N-methyl-t-butylglycine | Nmtbug |
| D-tyrosine | Dtyr | L-norleucine | Nle |
| D-valine | Dval | L-norvaline | Nva |
| D-α-methylalanine | Dmala | α-methyl-aminoisobutyrate | Maib |
| D-α-methylarginine | Dmarg | α-methyl-y-aminobutyrate | Mgabu |
| D-α-methylasparagine | Dmasn | α-methylcyclohexylalanine | Mchexa |
| D-α-methylaspartate | Dmasp | α-methylcylcopentylalanine | Mcpen |
| D-α-methylcysteine | Dmcys | α-methyl-α-napthylalanine | Manap |
| D-α-methylglutamine | Dmgln | α-methylpenicillamine | Mpen |
| D-α-methylhistidine | Dmhis | N-(4-aminobutyl)glycine | Nglu |
| D-α-methylisoleucine | Dmile | N-(2-aminoethyl)glycine | Naeg |
| D-α-methylleucine | Dmleu | N-(3-aminopropyl)glycine | Norn |
| D-α-methyllysine | Dmlys | N-amino-α-methylbutyrate | Nmaabu |
| D-α-methylmethionine | Dmmet | α-napthylalanine | Anap |
| D-α-methylornithine | Dmorn | N-benzylglycine | Nphe |
| D-α-methylphenylalanine | Dmphe | N-(2-carbamylethyl)glycine | Ngln |
| D-α-methylproline | Dmpro | N-(carbamylmethyl)glycine | Nasn |
| D-α-methylserine | Dmser | N-(2-carboxyethyl)glycine | Nglu |
| D-α-methylthreonine | Dmthr | N-(carboxymethyl)glycine | Nasp |
| D-α-methyltryptophan | Dmtrp | N-cyclobutylglycine | Ncbut |
| D-α-methyltyrosine | Dmty | N-cycloheptylglycine | Nchep |
| D-α-methylvaline | Dmval | N-cyclohexylglycine | Nchex |
| D-N-methylalanine | Dnmala | N-cyclodecylglycine | Ncdec |
| D-N-methylarginine | Dnmarg | N-cylcododecylglycine | Ncdod |
| D-N-methylasparagine | Dnmasn | N-cyclooctylglycine | Ncoct |
| D-N-methylaspartate | Dnmasp | N-cyclopropylglycine | Ncpro |
| D-N-methylcysteine | Dnmcys | N-cycloundecylglycine | Ncund |
| D-N-methylglutamine | Dnmgln | N-(2,2-diphenyl ethyl)glycine | Nbhm |
| D-N-methylglutamate | Dnmglu | N-(3,3-diphenylpropyl)glycine | Nbhe |
| D-N-methylhistidine | Dnmhis | N-(3-guanidinopropyl)glycine | Narg |
| D-N-methylisoleucine | Dnmile | N-(1-hydroxyethyl)glycine | Nthr |
| D-N-methylleucine | DnmleuD | N-(hydroxyethyl))glycine | Nser |
| D-N-methyllysine | nmlys | N-(imidazolylethyl))glycine | Nhis |
| N-methylcyclohexylalanine | Nmchexa | N-(3-indolylyethyl)glycine | Nhtrp |
| D-N-methylornithine | Dnmorn | N-methyl-γ-aminobutyrate | Nmgabu |
| N-methylglycine | Nala | D-N-methylmethionine | Dnmmet |
| N-methylaminoisobutyrate | Nmaib | N-methylcyclopentylalanine | Nmcpen |
| N-(1-methylpropyl)glycine | Nile | D-N-methylphenylalanine | Dnmphe |
| N-(2-methylpropyl)glycine | Nleu | D-N-methylproline | Dnmpro |
| D-N-methyltryptophan | Dnmtrp | D-N-methylserine | Dnmser |
| D-N-methyltyrosine | Dnmtyr | D-N-methylthreonine | Dnmthr |
| D-N-methylvaline | Dnmval | N-(1-methylethyl)glycine | NvalNman |
| γ-aminobutyric acid | Gabu | N-methyla-napthylalanine | ap |
| L-t-butylglycine | Tbug | N-methylpenicillamine | Nmpen |
| L-ethylglycine | Etg | N-(p-hydroxyphenyl)glycine | Nhtyr |
| L-homophenylalanine | Hphe | N-(thiomethyl)glycine | Ncys |
| L-α-methylarginine | Marg | penicillamine | Pen |
| L-α-methylaspartate | Masp | L-α-methylalanine | Mala |
| L-α-methylcysteine | Mcys | L-α-methylasparagine | Masn |
| L-α-methylglutamine | Mgln | L-α-methyl-t-butylglycine | Mtbug |
| L-α-methylhistidine | Mhis | L-methylethylglycine | Metg |
| L-α-methylisoleucine | Mile | L-α-methylglutamate | Mglu |
| L-α-methylleucine | Mleu | L-α-methylhomophenylalanine | Mhphe |
| L-α-methylmethionine | Mmet | N-(2-methylthioethyl)glycine | Nmet |
| L-α-methylnorvaline | Mnva | L-α-methyllysine | Mlys |
| L-α-methylphenylalanine | Mphe | L-α-methylnorleucine | Mnle |
| L-α-methylserine | Mser | L-α-methylornithine | Morn |
| L-α-methyltryptophan | Mtrp | L-α-methylproline | Mpro |
| L-α-methylvaline | Mval | L-α-methylthreonine | Mthr |
| N-(N-(2,2-diphenylethyl) | Nnbhm | L-α-methyltyrosine | Mtyr |
| carbamylmethyl)glycine | | L-N-methylhomophenylalanine | Nmhphe |
| 1-carboxy-1(2,2-diphenyl- | Nmbc | N-(N-(3,3-diphenylpropyl) | Nnbhe |
| ethylamino)cyclopropane | | carbamylmethyl)glycine | |

The “X” amino acid residues flanking -I- may be selected to correspond to the amino acid residues which flank -I¹³⁰ (i.e. the I at P1 or IGFBP-1) of human IGFBP-1 in nature, or they may be non-native amino acids (e.g. selected from homo- or heteropolymers of G, A, I, V, L etc).

A preferred representative of such peptide may be the octapeptide WDAISTYD or a fragment thereof which retains I, more particularly which retains the I corresponding or equivalent to I¹³¹ or I at P1 of IGFBP-1.

Representative fragments thus include: WDAI, DAI, DAIS, WDAIS, DAIST, WDAIST, DAISTY, WDAISTY, DAISTYD, AIS, AIST, AISTY, AISTYD.

It is believed that such peptides are novel therapeutic agents and accordingly in a further aspect the present invention provides a peptide as defined above for use in therapy. Most notably, in its broadest aspect, such a peptide may be defined as having from 4 to 20 amino acids, comprising an I residue (particularly an I residue corresponding or equivalent to I¹³¹ or I at P1 of IGFBP-1), and being capable of acting as a proteolytic substrate for azurocidin.

Also part of the invention is a pharmaceutical composition comprising a peptide of the invention as defined above, together with at least one pharmaceutically acceptable diluent or excipient.

As noted above, a preferred peptide according to this aspect of the invention is WDAISTYD or an -I- containing fragment thereof. It is believed that such a peptide is novel and accordingly a further aspect of the invention provides a peptide having or comprising the amino acid sequence WDAISTYD or a fragment thereof which retains I, more particularly which retains the I corresponding or equivalent to I¹³¹ or I at P1 of IGFBP-1, wherein said peptide or fragment thereof is capable of acting as a proteolytic substrate for azurocidin.

In an alternative embodiment of the invention, the modulator enhances the interaction between azurocidin and IGFBP-1. Such a modulator may include an agonist of the interaction. In a preferred embodiment, a modulator which enhances the interaction is aprotinin.

Aprotinin is a protein well known in the art and marketed as Trasylol by Bayer Pharmaceuticals. Aprotinin has hence been defined in the patent literature, for example in WO 89/10374, U.S. Pat. No. 5,591,603, EP 375718, EP 339942 and EP 487591. (Insofar as all the listed patent specifications refer to aprotinin and fragments or derivatives of analogues thereof, they are incorporated by reference herein.) It has been used or proposed for use therapeutically for a number of different indications, including anti-fibrinolytic activity. Much has been published on its use as a serine protease inhibitor, for example in coronary artery bypass graft surgery (Am J Health Syst Pharm 2005 62:S9. Its sequence and that of its cDNA is available at http://www.ncbi.nlm.nih.gov/entrez/viewer.fcgi?db=protein&val=48475058. Furthermore, details of fragments, variants and modifications etc. of aprotinin are widely published in the literature.

We have surprisingly observed that aprotinin can enhance the interaction, increasing IGFBP-1 proteolysis. It is known that aprotinin, a serine protease inhibitor, can bind to azurocidin, and the observation that it is not a protease inhibitor in these circumstances is unexpected.

As for azurocidin above, functionally equivalent variants, fragments or derivatives of aprotinin may be used, and the term “aprotinin” as used herein encompasses any such fragments, variants or derivatives and includes all known form of aprotinin as described in the literature.

As described for azurocidin above, the modulators which enhance or reduce the interaction between azurocidin and IGFBP-1 can be administered as a pharmaceutical composition. The present invention therefore also provides a pharmaceutical composition comprising a modulator of azurocidin and IGFBP-1 interaction together with at least one pharmaceutically acceptable carrier, diluent or excipient.

In a preferred embodiment of this aspect, the modulator is an agent capable of blocking the interaction between azurocidin and IGFBP-1.

The modulator in such compositions may comprise from 0.05% to 99% by weight of the formulation. Pharmaceutical compositions comprising the modulators described herein for use in therapy may be formulated by techniques known in the art, such as described above with respect to azurocidin compositions.

Appropriate dosages and dosage forms may readily be determined by the skilled practitioner, according to principles and procedures well known in the art. Where the blocking modulators are concerned, they may be administered so as to be “in excess” of endogenous IGFBP-1: it may be necessary to achieve tissue levels as high as 10 μM.

As noted above, the modulators of the invention may be used to treat any condition which is associated with IGFBP-1 and/or IGF levels and/or activity.

In particular enhancers of the interaction may be used to treat conditions associated with elevated IGFBP-1 levels; enhancers will promote degradation of endogenous IGFBP-1 and hence may be used in the treatment of any condition which is responsive to, or would benefit from, a reduction in IGFBP-1 levels, for example to promote IGF levels, e.g. the growth promoting effects of IGF, or to reduce harmful effects of elevated or excessive IGFBP-1 levels.

Such conditions associated with elevated IGFBP-1 levels include the conditions listed above, in relation to azurocidin therapy, including in particular conditions associated with hepatic insulin resistance, as well as renal disease, and growth impairment in children with diabetes. Particular mention may be made also of the specific conditions listed above which are subsumed under the general heading “conditions associated with hepatic insulin resistance”. However, IGFBP-1 is known to be elevated in conditions such as septic shock, and in critical illness and intensive care patients irrespective of hepatic insulin resistance. Accordingly, in this aspect of the invention, an enhancer of the azurocidin-IGFBP-1 interaction may be used in the treatment of any such condition whether or not there is hepatic insulin resistance. Thus conditions such as septic shock, trauma (e.g. following injury or surgery, particularly major surgery), critical illness, intensive care are covered in general, as is any catabolic condition associated with elevated IGFBP-1 levels.

Accordingly, in a preferred aspect the present invention also provides the use of a modulator which enhances the interaction between azurocidin and IGFBP-1 in the manufacture of a medicament for the treatment of a condition associated with elevated levels of IGFBP-1.

More preferably, the invention covers the use of aprotinin in the manufacture of a medicament for the treatment of a condition associated with elevated levels of IGFBP-1.

A modulator which blocks the interaction may be used to treat any condition associated with an excessive or unwanted level or activity of IGF; by blocking the interaction, degradation of IGFBP-1 is prevented and hence it is available to bind IGF-1 and reduce its bioavailability. Thus free IGF levels and hence IGF activity may be reduced. Such a condition may accordingly be any condition in which it is desirable to maintain or increase IGFBP-1 levels. Furthermore, as explained above since the proteolytic fragments of IGFBP-1 may themselves show IGF-promoting activity, it may be desirable to prevent the formation of such fragments in any condition in which IGF activity is undesirable, or where it is beneficial to reduce or minimise IGF activity.

A blocking modulator may thus be used to treat (or in the treatment of), for example, cancer, particularly tumours which are IGF-dependent (e.g. IGF-I dependent).

Azurocidin is an inflammatory mediator and is released in response to infection or injury. Inflammation which occurs when, for example, a patient is suffering from a condition where IGF activity is undesirable (e.g. cancer or arteriosclerosis as mentioned above) may accordingly have undesirable and harmful consequences in raising IGF levels (mediated by the action of azurocidin in cleaving IGFBP-1). Accordingly such patients would benefit from blocking the interaction in order to prevent or reduce IGFBP-1 cleavage.

A blocking modulator may therefore be useful in the treatment of any condition associated with inflammation, in which IGF activity contributes to the disease process (i.e. the condition). This would include, as mentioned above cancer, which may include any tumour, and cancer such as multiple myeloma and Hodgkin's lymphoma (which are haematological malignancies that are characterised by a local inflammatory response) and atherosclerosis and any other condition associated with excessive or unwanted cell proliferation.

Accordingly, in a preferred embodiment, the present invention therefore provides use of a modulator which blocks or reduces the interaction between azurocidin and IGFBP-1 in the manufacture of a medicament for the treatment of a condition associated with unwanted and/or excessive IGF activity.

Advantageously such a condition is preferably inflammation, particularly when associated with a pathological condition (or disease) in which IGF activity is undesirable (e.g. wherein IGF activity contributes to the pathological condition or disease).

As noted above preferred blocking modulators include anti-azurocidin antibodies or peptides which compete with IGFBP-1 as substrates for azurocidin.

Another therapeutic utility arises from the novel interaction between azurocidin and IGFBP-1. As noted above, proteolytic fragments of IGFBP-1 may retain the RGD-dependent effects of intact IGFBP-1 i.e. retain the ability of IGFBP-1 to interact with the integrin receptor. Accordingly, as for intact IGFBP-1 itself, proteolytic fragments thereof may have therapeutic utility in promoting wound healing. Thus, for example, such fragments may be applied topically or locally to a wound or injury to promote heating. The C-terminal fragment, IGFBP-1¹³¹⁻²³⁴ retains the RGD, integrin-binding sequence. This may further degrade to IGFBP-1¹⁴²⁻¹¹⁴ which also has the RGD, integrin-binding sequence. C-terminal fragments of IGFBP-1 (in particular IGFBP-1¹³¹⁻²³⁴) have lower affinity for IGFs (and therefore will promote IGF action) as well as the ability to bind to the fibronectin receptor and stimulate cell migration. Such properties underlie a beneficial effect in wound healing. Both N- and C-terminal fragments of IGFBP-1 are able to bind to IGF.

In a further aspect, the present invention accordingly provides a combination of azurocidin and IGFBP-1 for use in therapy. More particularly this therapy is wound healing.

The azurocidin may conveniently be administered together with IGFBP-1 in a single composition. Thus, the invention also provides a pharmaceutical composition comprising azurocidin and IGFBP-1, together with at least one pharmaceutically acceptable carrier, excipient or diluent.

Conveniently, such a composition may be a topical composition as discussed above.

It is not however necessary that the azurocidin be administered together with the IGFBP-1 in a single composition, and they may be administered separately, in separate compositions at the same time or different times.

Accordingly in a related aspect, the present invention provides a product containing azurocidin and IGFBP-1 as a combined preparation for simultaneous, separate or sequential use in wound healing.

Alternatively viewed, this aspect of the invention provides use of azurocidin together with IGFBP-1 for the preparation of a medicament for use in wound healing.

The invention will now be described in more detail in the following non-limiting Examples which show, with reference to the following drawings:

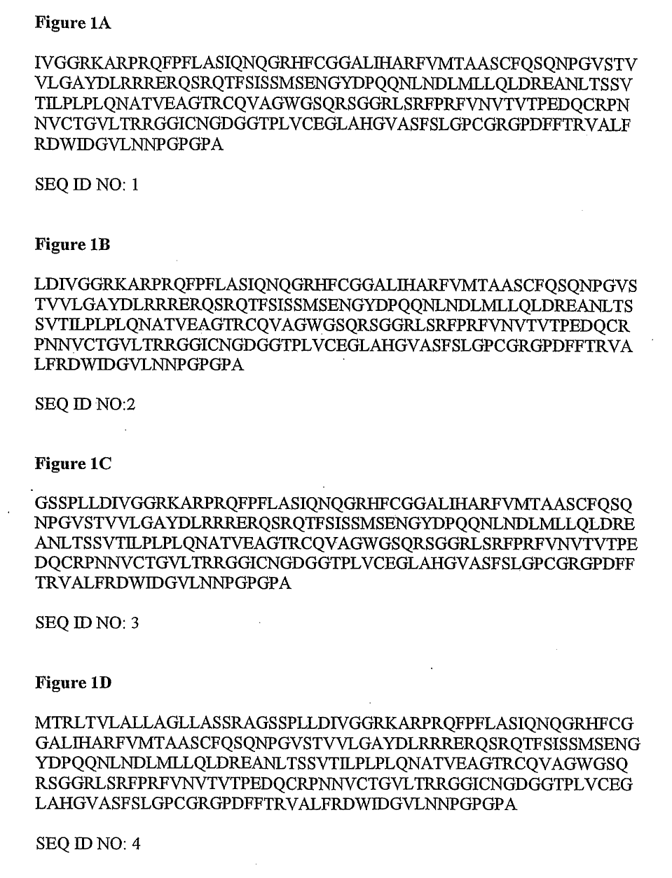

FIG. 1 shows protein sequences for azurocidin. FIG. 1A shows SEQ ID NO. 1 which is the fully processed human protein sequence of azurocidin; FIG. 1B shows SEQ ID NO. 2 which is huma preazurocidin; FIG. 1C shows SEQ ID NO. 3 which is human preproazurocidin; and FIG. 1D shows SEQ ID NO. 4 which is the full product encoded by the cDNA of SEQ ID NO. 5 i.e. is azurocidin before any processing.

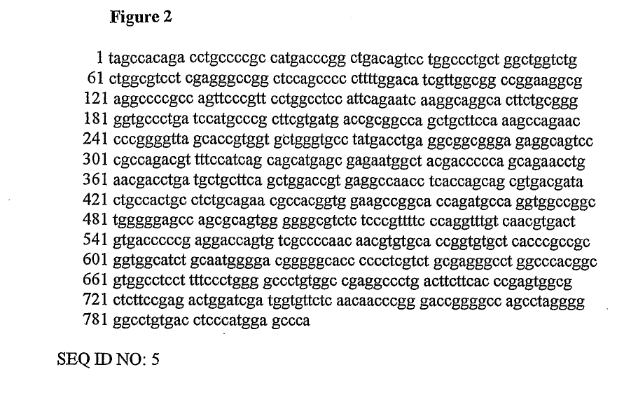

FIG. 2 shows SEQ ID NO. 5 which shows the human coding sequence for azurocidin;

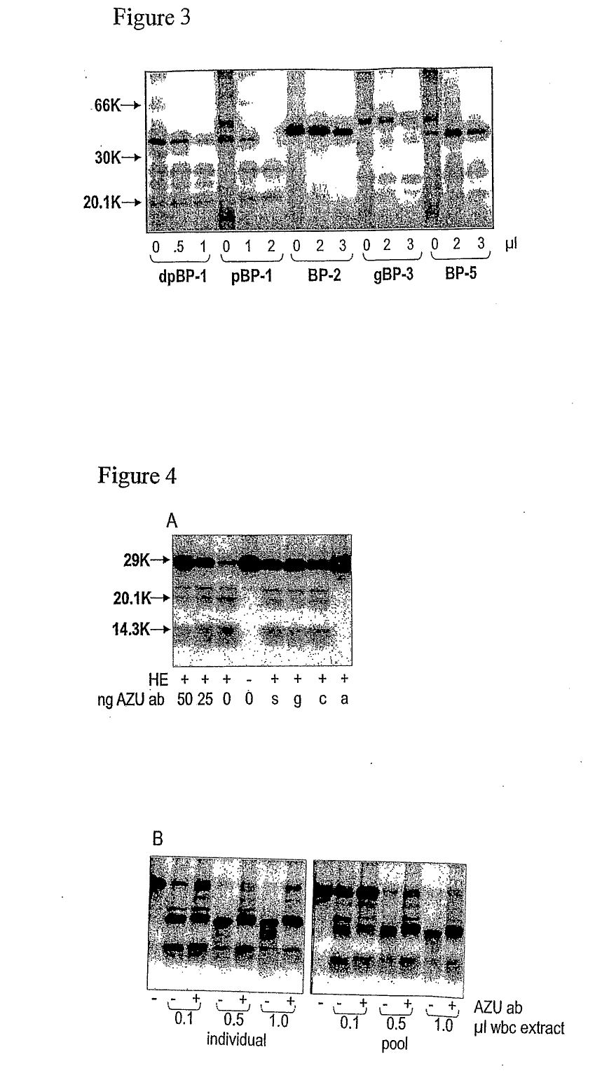

FIG. 3 shows the presence of IGFBP-1-specific protease activity in human urine. Pure IGFBPs, 10 ng, were incubated with the indicated volumes of urine under neutral conditions for 2 h at 37 C. No IGF or IGFBP immunoreactivity was present in the urine. After incubation, IGF-binding proteins were affinity labelled with radioiodinated IGF-I and subjected to SDS-PAGE. The gel was dried and autoradiographed. Urine from this individual preferentially cleaved native IGFBP-1, both the highly phosphorylated, and the dephosphorylated (dp) isoform. One μl of urine cleaved more than 50% of each IGFBP-1 isoform, while 2 μl had minimal effect on IGFBP-2, glycosylated (g) IGFBP-3, IGFBP-5 and, not shown here, IGFBP-4 and IGFBP-6;

FIG. 4 shows azurocidin-dependent IGFBP-1 protease activity. Recombinant phosphorylated IGFBP-1 was used as substrate and incubations were carried out under neutral conditions for 2 h at 37 C. IGFBP-1 fragments were separated on SDS-PAGE, and detected by ECL as described in the methods. Panel A: Incubations with 0.25 μL eluate (HE) from heparin affinity chromatography (50 μl urine equiv.) in the presence and absence of anti-azurocidin antibody; and 1 μl HE of the loading material (L), and the flow through after affinity chromatography on protein G columns without (G) and with bound anti-chymase (C) or anti-azurocidin (A) antibodies. Panel B: Incubations in the presence and absence of 25 ng anti-azurocidin antibody with increasing volumes of white cell extract from the individual with a specific IGFBP-1 protease in urine compared to the white cell extract pooled from 6 healthy subjects;

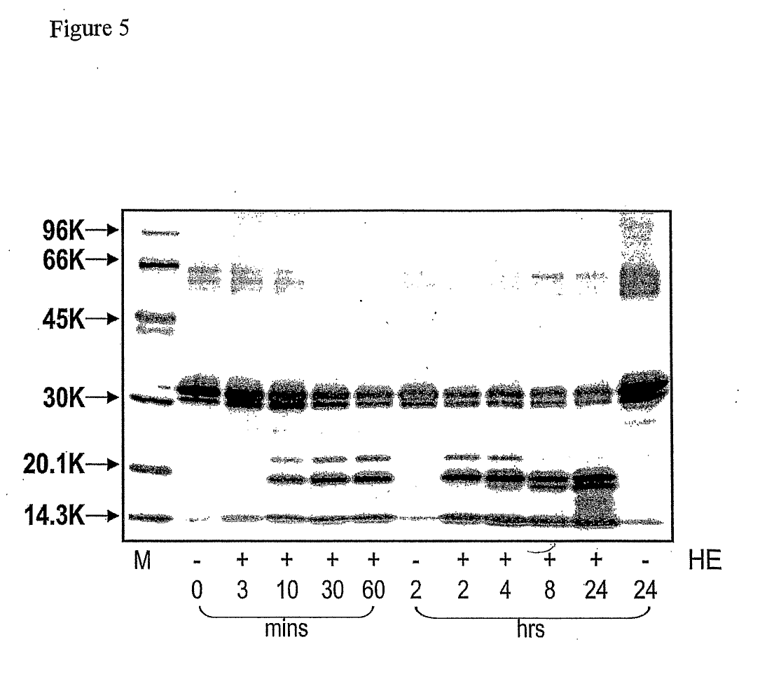

FIG. 5 shows phosphorylated IGFBP-1 is cleaved rapidly by azurocidin. Recombinant IGFBP-1 was incubated with the eluate from heparin affinity chromatography (HE) with a ratio of azurocidin:BP-1 of 1:3,500. After the times indicated, proteins were detected by silver staining after SDS-PAGE;

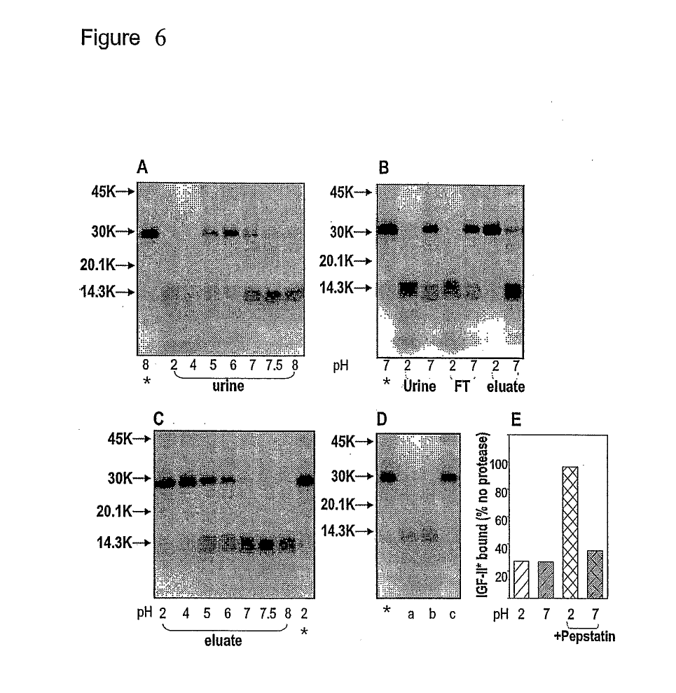

FIG. 6 shows separation of acid-activated and neutral IGFBP-1 protease activity in human urine. A-D: Radioiodinated recombinant npIGFBP-1 was subjected to 14% SDS-PAGEalone (*) or after proteolysis for 2 h at 37 C with 0.5 μl urine or 0.25 μl eluate, and the fragments detected by autoradiography. Molecular mass markers are indicated by the arrows. A: pH-dependence of urinary IGFBP-1 protease activity. B: Urine, flowthrough (FT) from the heparin affinity column, and eluate at pH 2 and 7. C: pHdependence of eluate from heparin affinity chromatography. D: Acid-activated protease activity is destroyed by neutralization. Radioiodinated pIGFBP-1 was incubated without (*) and with 0.5 μl urine for 2 h at 37 C at pH 2. Prior to incubation the urine was either untreated (lane a), adjusted to pH 2 for 15 min (lane B) or adjusted to pH 2 for 15 min and then to pH 7 for a further 15 min (lane C). E: Effect of pepstatin. Biotinylated recombinant npIGFBP-1 10 ng was bound to streptavidin coated plates and incubated with 5 μl urine for 2 h at 37 C in the presence and absence of pepstatin A 10 μM at pH 2 and 7. The wells were washed, incubated with radioiodinated IGF-II for 2 h at 22 C, washed, and the bound counts detected;

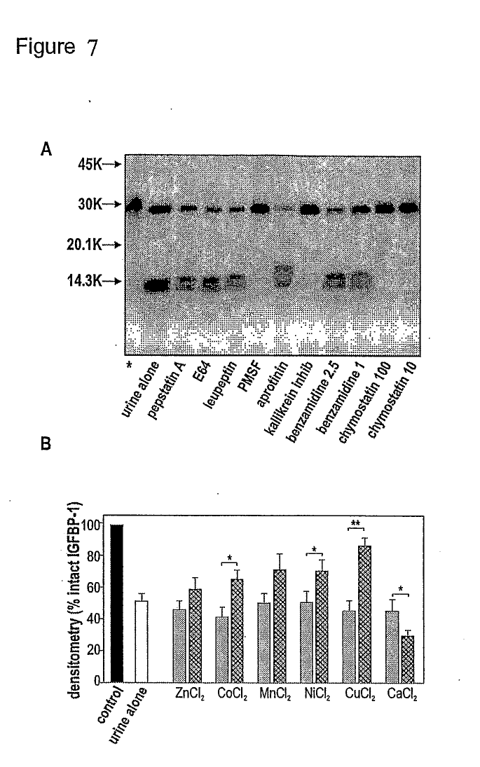

FIG. 7 shows effect of protease inhibitors and divalent cations on urinary protease activity at neutral pH Radioiodinated recombinant npIGFBP-1 was incubated alone (*) or in the presence of 0.5 μL urine, in the absence and presence of protease inhibitors or metal cations at pH 7 for 2 h at 37 C, then subjected to 14% SDS-PAGE and the fragments detected by autoradiography. A: Effect of 100 μM pepstatin A, 10 μM E64, 2.5 mM leupeptin, 10 mM PMSF, 100 μM aprotinin, 2.5 mM kallikrein inhibitor and benzamidine (mM) and chymostatin (μM). Molecular mass markers are indicated by the arrows. B: Densitometry measurements of intact IGFBP-1. The results are the mean±SEM, pooled from 6-8 experiments. The effects of 10 mM or each cation are displayed in the hatched bars alongside the effect of urine in the absence of cation (light grey bars). The mean effect of urine pooled from all the experiments is shown in the open bar. *, P<0.05; ** P<0.01 (paired t-test);

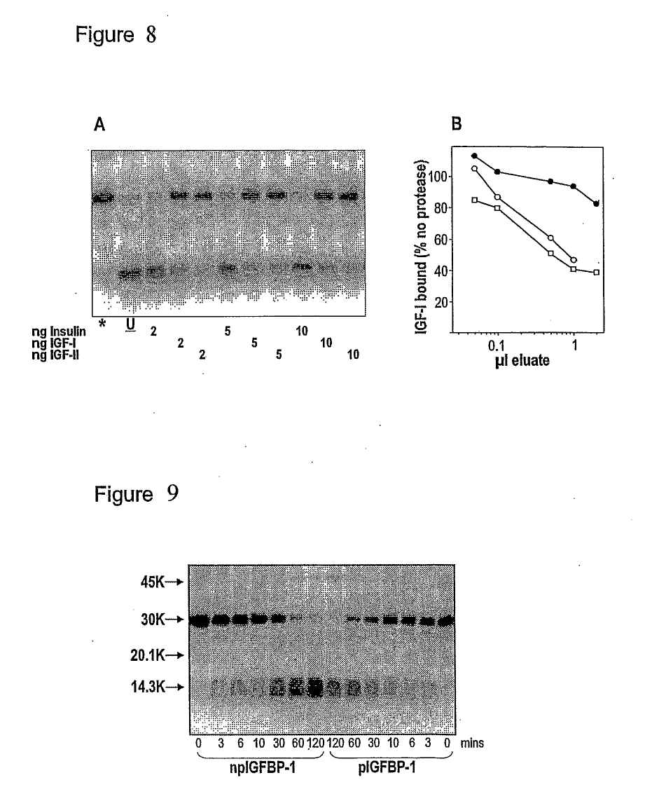

FIG. 8 shows IGFs partially inhibit azurocidin-dependent protease activity A: Radioiodinated recombinant npIGFBP-1 was first incubated with increasing concentrations of peptides at pH 7 for 30 min at 22 C. Urine, 0.5 μl, was added and the incubation continued for 2 h at 37 C. The fragments were separated by 14% SDS-PAGE and detected by autoradiography. Molecular mass markers are indicated by the arrows. B: Biotinylated recombinant pIGFBP-1 was bound to streptavidin coated plates and then incubated without (open squares) or with 100 ng IGF-I (closed circles) or des(1-3)IGF-I (open circles). Wells were then incubated with increasing concentration of partially purified azurocidin at pH for 2-h at 37 C. After acidification of all wells, including controls, with 1 M HCl to dissociate bound peptides, radioiodinated IGF-I was added for 2 h at 22 C, unbound tracer was removed by washing, and the bound counts determined;

FIG. 9 shows time course comparing proteolysis of npIGFBP-1 and pIGFBP-1 Radioiodinated recombinant npIGFBP-1 and pIGPBP-1, representing approx 0.35 ng/lane were incubated with 0.5 μl urine at 37 C for the times indicated. Fragments were separated by 14% SDS-PAGE and detected by autoradiography. Molecular mass markers are indicated by the arrows;

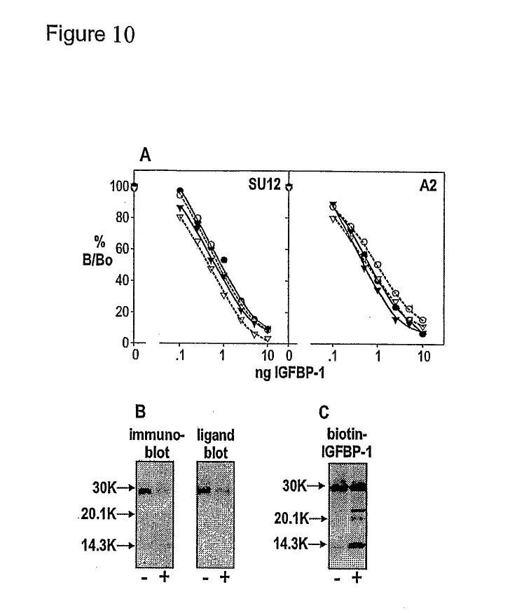

FIG. 10 shows effect of proteolysis on IGFBP-1 immunoreactivity and ligand binding ability of native IGFBP-1 A: Radioimmunoassay for IGFBP-1 using 2 polyclonal antibodies, SU12 and A2. Increasing concentrations of HepG2-purified IGFBP-1, phosphorylated (triangles) and dephosphorylated (circles) were incubated without (open symbols) and with (closed symbols) 5 μl urine for 5 h at 37 C before radioimmunoassay. Results are expressed as specific binding as a percentage of tracer binding in the absence of IGFBP-1. There was no effect of urine on non-specific binding. Radioiodinated recombinant npIGFBP-1 was used as tracer. B: HepG2 purified phosphorylated IGFBP-1, 100 ng was incubated without (−) and with (+) 0.5 μl urine for 60 min at 37 C, subjected to 14% SDS-PAGE, and transferred to PVDF membranes. Immunoblotting was carried out with anti-IGFBP-1 antibody, SU12 1:1 000 and ligand blotting, with radioiodinated IGF-II. Molecular mass markers are indicated by the arrows. C: Recombinant pIGFBP-1 was biotinylated and incubated without (−) and with (+) 0.25 μl eluate from heparin affinity chromatography for 2 h at 37 C. IGFBP-1 fragments were separated on 14% SDS-PAGE and detected by ECL;

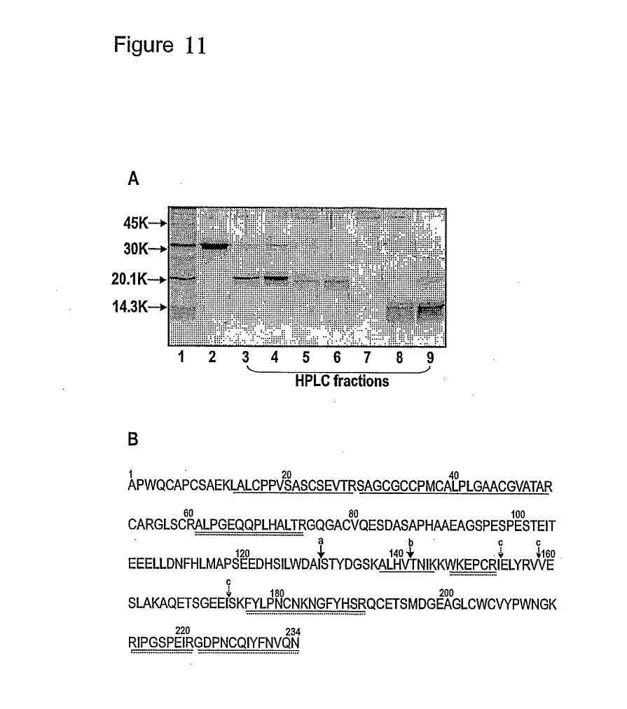

FIG. 11 shows IGFBP-1 fragments generated by azurocidin-dependent proteolysis. A: Recombinant npIGFBP-1 was incubated without (−) and with (+) 0.25 μl partially purified azurocidin in a protease:substrate ratio of 1:3 500 for 2 h at 37 C. The fragments were purified on HPLC. N-, purified fragments with the N-terminal sequence of IGFBP-1, APWQCAP-, c-, small fragment with the sequence STYDGSK-, C-, purified C-terminal fragments with the sequence TNIKKWK-. Samples were separated on 12% SDS-PAGE and visualised by silver staining. Molecular mass markers are indicated by the arrows. B: Summary of sequence analysis of IGFBP-1 fragments after azurocidin-dependent proteolysis. N-terminal sequence analysis was performed on fragments purified by HPLC on 4 separate occasions. C-terminal sequence analysis was perform on one HPLC run. Cleavage sites are indicated by the arrows. a, the primary cleavage site present in 4 runs. b, a secondary cleavage site, present in 3 runs. c, cleavage sites, each identified in only one of the four runs. MALDI-MS analysis was performed on tryptic digests of fragments excised from SDS-PAGE. Continuous lines indicate sequences present in the approx 20 kDa fragments. Broken lines indicate those in the 14 kDa forms;

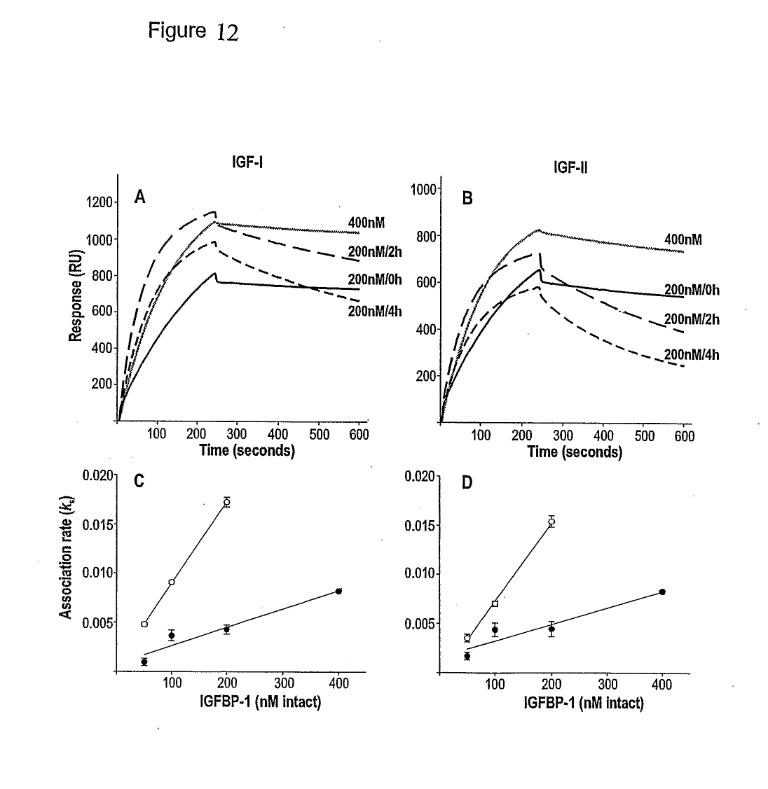

FIG. 12 shows BIAcore analysis of intact and proteolyzed pIGFBP-1 over IGF-I and IGF-II biosensor surfaces. Recombinant pIGFBP-1 was analyzed across IGF-I (A, C) and IGF-II (B, D) biosensor surfaces. A concentration range of 50, 100, 200, 400 nM was analyzed at a flow rate of 10 μl/min. A,B: Mean sensorgrams for 400 nM IGFBP-1 (intact, n=3) and 200 nM IGFBP-1, intact (0 h, n=4) and after 2-h (n=5) and 4-h (n=3) incubations with partially purified azurocidin in a protease:substrate ratio of 1:3 500 for 2 h at 37 C. C,D: The rate of association (ks), the absolute value of the slope (dRU/dt versus RU) over the linear initial association phase (21-120 s), is shown for pIGFBP-1, intact (closed circles), and after incubation with partially purified azurocidin for 2 h at 37 C (open circles); and

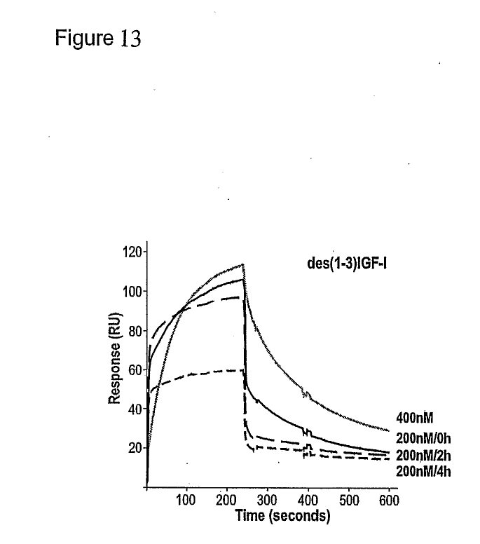

FIG. 13 shows BIAcore analysis of intact and proteolyzed pIGFBP-1 over des(1-3)IGFI. Recombinant pIGFBP-1 was analyzed across a des(1-3) IGF-I biosensor surfaces with a concentration range of 50, 100, 200, 400 nM, at a flow rate of 10 μl/min. The mean sensorgrams are shown for 400 nM IGFBP-1 (intact, n=3) and 200 nM IGFBP-1, intact (0 h, n=4) and after 2-h (n=5) and 4-h (n=3) incubations with partially purified azurocidin in a protease:substrate ratio of 1:3 500 for 2 h at 37 C.

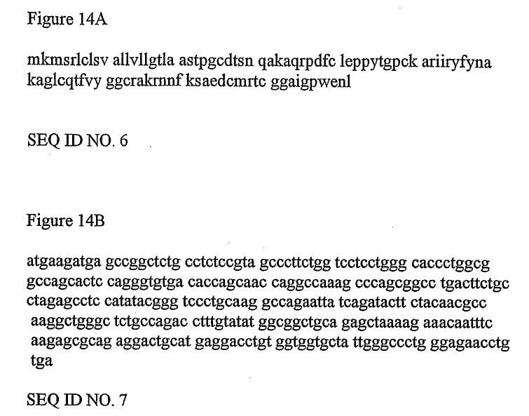

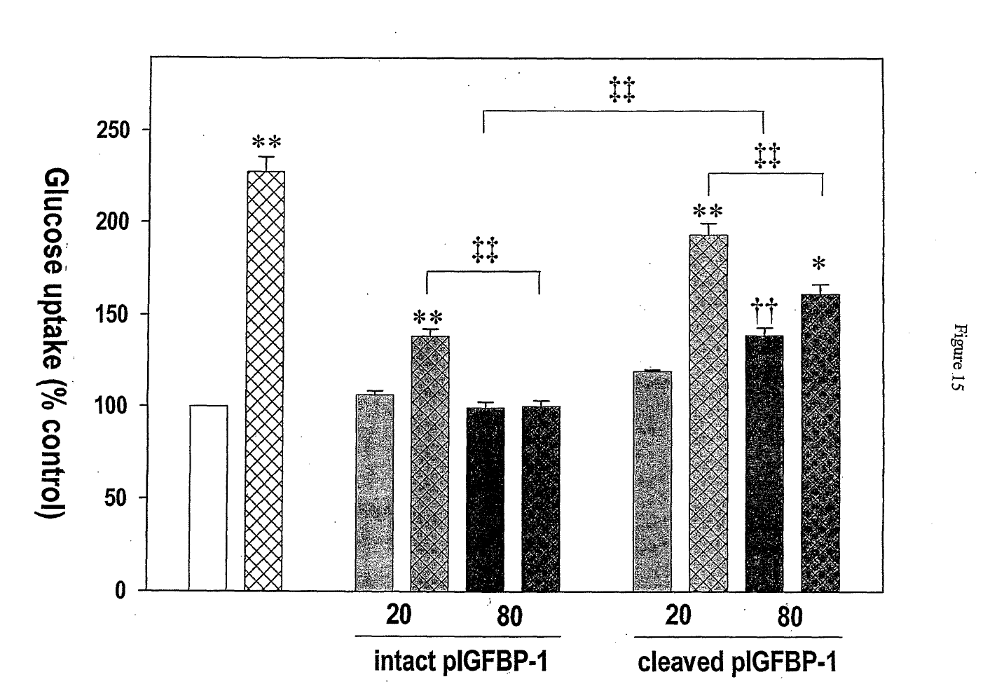

FIG. 14 shows aprotinin sequences. FIG. 14A shows the protein sequence of aprotinin (SEQ ID NO. 6) and FIG. 14B shows the coding sequence for aprotinin (SEQ ID NO. 7).

FIG. 15 Effect of proteolysis on pIGFBP-1 action on glucose uptake into primary human skeletal myotube culture

Primary human skeletal myotubes were incubated under serum-free conditions for 24 h. Glucose uptake was determined as described in the Methods. The results are the mean±SEM of four experiments using cells from different individuals each with 3 replicates; and are expressed as the percentage of uptake in the absence of IGF-II or IGFBP-1 for each. The effect of IGF-II is shown in the hatched bars. The effect of pIGFBP-1 on stimulation of glucose uptake by 10 ng/ml IGF-II was analyzed by 2-way ANOVA for each of the 20 ng/ml and 80 ng/ml concentrations; *, P<0.01, **, P<0.001, compared to no IGF-II; ††, P<0.001, compared to no IGFBP-1 or intact IGFBP-1. The effect of 20 ng/ml compared with 80 ng/ml IGFBP-1 on IGF-II-stimulated glucose uptake was compared in a separate 2-way ANOVA; ‡‡, significant difference between intact and proteolysed IGFBP-1, and a significant effect of concentration; each P<0.001, with no interaction.

### EXAMPLES

### Example 1

### IGFBP-1 Specific Proteolysis in Human Urine and Isolation and Identification of Azurocidin as an IGFBP-1 Protease

### Methods

All peptides used are human isoforms. Recombinant phosphorylated IGFBP-1 and recombinant IGF-I were kindly donated by Kabi Pharmacia (Sweden). Highly phosphorylated HepG2-purified IGFBP-1 and a dephosphorylated preparation were purchased from Sigma (Sweden). IGFBP-2, glycosylated IGFBP-3, IGFBP-4, and IGFBP-6 were from GroPep Limited (Adelaide, Australia), IGFBP-5 from Upstate (KELAB, Göteborg, Sweden). IGFBP-1 was biotinylated with sulfo-NHS-LC-LC-biotin (EZ-Link, Pierce), IGF-I was iodinated using lactoperoxidase and purified by HPLC. Recombinant azurocidin²⁷⁻²⁵⁰, purified from murine myeloma cells, and the anti-human azurocidin mouse monoclonal antibodies were from R&D Systems Europe Ltd (Abingdon, UK), while the anti-human chymase mouse monoclonal antibody was from Calbiochem-Novabiochem Corporation (San Diego, Calif.). Human urine and blood were collected with approval of the local Ethics Committee. A crude preparation of white cells was prepared by differential lysis of erythrocytes in buffer containing 155 mM ammonium chloride, 10 mM sodium bicarbonate, 0.1 mM EDTA. White cells were then lysed by 3 freeze-thaw cycles.

### Protease Assays

After incubation without and with protease in phosphate buffered saline, pH 7 at 37 C, pure IGFBPs were affinity labelled with [¹²⁵I]IGF-I using DSS (Pierce, Rockford, Ill.) and the fragments separated on 14% nonreducing SDS-PAGE. Gels were fixed, dried and analysed with the Fuji PhosphoImage program (Fuji Co., Stockholm, Sweden). Biotinylated IGFBP-1 and its fragments were separated on 14% SDS-PAGE under reducing conditions, transferred to PVDF (Biorad, Sundbyberg, Sweden), incubated with neutravidin-horseradish peroxidase (Pierce Biotechnology Inc, Rochester, Ill.) and detected by ECL (Amersham Biosciences, Uppsala, Sweden). Silver staining of proteins and fragments was performed after denaturing 12% PAGE under reducing conditions (SilverQuest and NuPAGE, Invitrogen, Stockholm, Sweden).

### Isolation of IGFBP-1 Protease

Heparin affinity chromatography was carried out in batches using heparin-agarose gel (Pharmacia). Urine was diluted 1:5 in 10 mM sodium phosphate, with 0.25 M NaCl buffer, pH 7 and incubated with gel for 30 min at 4 C. The gel was transferred to a column and washed with 10 times the gel volume of buffer. Heparin binding proteins were eluted in loading buffer containing 2M NaCl and the eluate was desalted, concentrated using a 10 kDa spin column and stored at −80 C. Concentrated material derived from 700 ml urine were applied to C4 reverse-phase HPLC, in 0.1% TFA on an acetonitrile gradient of 0-100%. Immunoaffinity chromatography was performed using monoclonal anti-azurocidin antibody attached to protein G Sepharose (Sigma-Aldrich, Sweden). In brief, antibody bound gel was incubated with the eluate from heparin affinity chromatography for 2 h at 22 C, and transferred to a Bio-Spin chromatography column (BioRad). The flow-through was collected for analysis of protease activity. As controls, an additional column was prepared with a monoclonal antibody against human mast cell chymase and a second column with no bound antibody.

### Sequence Analysis

The primary cleavage site in IGFBP-1 was determined by HPLC-separation of phosphorylated IGFBP-1 after incubation with heparin eluate, and HPLC purification of fragments, followed by N- and C-terminal sequencing. LC- and MS/MS MALDI mass spectrometry was carried out after in-gel digestion with trypsin (ref 1). N- and C-terminal sequencer degradations were performed on HPLC purified proteins in ABI instruments as described (refs 2 and 3), From this analysis, the quantity of azurocidin quantity in urine was estimated to be 25 pmol/100 ml.

### Results

Human urine was screened for IGFBP-1 protease activity which resulted in the detection of one individual with a specific IGFBP-1 (FIG. 3). The individual was a 73 year old woman with a long history of atopy with inflammatory skin lesions and eosinophilia who had a 7 year history of monoclonal gammopathy which over the next three years progress to meet the criteria for smouldering myeloma.

Urine was subjected to heparin affinity chromatography and IGFBP-1 protease activity was found to be present in the desalted eluate (FIG. 4A).

Silver staining of the eluate revealed three proteins that were identified by mass spectrometry as myeloperoxidase, lactoferrin and azurocidin (heparin-binding protein/CAP37), proteins that are abundant in polymorphonuclear leukocytes. The presence of myeloperoxidase and azurocidin was confirmed by N-terminal sequence analysis after reverse-phase HPLC. The proteolytic effect of the heparin affinity eluate on IGFBP-1 was completely abolished in the presence of a monoclonal antibody to azurocidin (FIG. 4A). Furthermore, using this antibody for immunoaffinity chromatography, specific extraction of azurocidin resulted in complete loss of proteolytic activity from the heparin-purified material. Extracts of white cells from the subject and pooled from 6 healthy women also contained IGFBP-1 proteolytic activity that was partially inhibited by the azurocidin antibody (FIG. 4B).

Azurocidin is a member of the Serprocidin family of serine protease homologues. However, to date it has been regarded as proteolytically inactive since two amino acids of the classical catalytic triad, including the “active-site” serine, are replaced. Recombinant human azurocidin²⁷⁻²⁵⁰, produced in mouse myeloma cells, had no effect on IGFBP-1 (data not shown). After HPLC-passage our purified azurocidin was also without effect. In fact, exposure of our heparin affinity-purified active material to the 0.1% TFA in the HPLC buffer, followed by lyophilization and reconstitution in assay buffer abolished the IGFBP-1 proteolytic activity. Furthermore, reduction with dithiothreitol of our native material resulted in loss of activity (data not shown).

The primary cleavage site of IGFBP-1, determined by N- and C-terminal sequence analysis of HPLC-purified fragments, was at Ile¹³⁰-Ser¹³¹. Present in the highly variable central domain of IGFBPs, the region around this cleavage site is highly conserved in IGFBP-1 between species, but not found in the other IGFBPs. The serine residue at the site is not reported to be significantly phosphorylated, whereas residues Ser¹⁰¹, ser¹¹⁹, Ser¹⁶⁹ are sites for phosphorylation which increase the affinity for IGF-I, and confer resistance to previously described proteases.

Azurocidin-dependent proteolysis of phosphorylated IGFBP-1 was seen within minutes, using a protease:substrate molar ratio of 1:3,500 (FIG. 5). This contrasts to other studies in which long term (>18-h) incubations with protease:substrate ratios of at least 1:10 for MMP-3, -9 and -26 were required to cleave non-phosphorylated IGFBP-1. The proteolytic effect of azurocidin is noticeable at a nanomolar concentration, substantially less than that required for its other effects. For example monocyte chemotaxis requires a micromolar level.

### Example 2

### Characterisation of Azurocidin Proteolytic Activity and the IGFBP-1 Fragments Generated by its Action

### Methods

In addition to the methods described in Example 1, the following methods were used.

Protease inhibitors were purchased from Sigma. des(1-3)IGF-I was from GroPep (Adelaide, Australia).

### Protease Assays

IGFBP-1 was radioiodinated with ¹²⁵I using the chloramine T method. After incubation without and with protease in phosphate buffered saline, pH 7 at 37 C, fragments were separated on 14% non-reducing SDS-PAGE, the gel dried, and visualized by autoradiography.

IGFBP-1 was biotinylated with sulfo-NHS-LC-LC-biotin and bound to streptavidin coated plates, then incubated without and with protease in phosphate buffered saline, pH 7 at 37 C. After washing, IGF-binding fragments were determined by incubation with radioiodinated IGF-I for 2 h at 22 C. Unbound tracer was removed by washing, and the bound counts determined.

Pure IGFBP-1 was incubated without and with protease in phosphate buffered saline, pH 7 at 37 C, and subjected to 12% SDS-PAGE. Proteins were transferred to nitrocellulose membranes, that were blocked with 0.25% Tween and then probed with radioiodinated IGF-II or SU12 antibody (1:1000).

### Radioimmunoassays

Two IGFBP-1 radioimmunoassays using rabbit polyclonal antibodies were used. One using antibody A2, provided by Prof Robert Baxter (The University of Sydney, Australia) at a dilution of 1:30 000, and the second using antibody SU12, raised within our own department, at a dilution of 1:9 000. For both assay we used non-phosphorylated IGFBP-1 as tracer. Samples were incubated 18 h at 4 C, and bound tracer counts separated using goat-anti-rabbit antiserum (Pharmacia).

### BIAcore Analysis

Instrument and reagents (buffers, chemicals for activating and deactivated) were purchased from BIAcore (Uppsala, Sweden). IGF-I, IGF-II and des(1-3)IGF-I were immobilized on the sensor chip by amine coupling and deactivated with 0.1M ethanolamine. A reference flow cell was also prepared to correct for background and bulk refractive index contribution. After equilibration buffer to achieve a stable baseline, binding experiments was at 22 C in 2 separate experiments with the samples injected in random order. 40 μl of each sample was injected over the sensor surface at a rate of 10 μl/min (association period), followed by a 6 min wash with buffer (dissociation period). The sensor surface was regenerated with 20 μl of 100 mM HCl and washed for 6 min before the next sample. We used a 1:1 Langmuir binding interaction model for kinetic analysis using the BIAcore software and GraphPad Prism (GraphPad Software Inc, San Diego, Calif.).

### Results

Altering the pH revealed 2 regions of IGFBP-1 proteolysis in the urine; one at pH 2-4 and another at pH 7-8 (FIG. 6A). Speculating that the acidic activity might represent pepsin, or a pepsin-like protease, the following experiments were undertaken. Firstly the effect of protease inhibitors was determined, and it was found that pepstatin inhibits the acidactivated, and not the neutral activity (FIG. 6E). Secondly, acid-activated urine was neutralised, and re-acidified, a process known to destroy pepsin. This resulted in complete loss of IGFBP-1 protease activity at pH 2 (FIG. 6D). These observations support the hypothesis that pepsinogen or a related protease is present in the urine of this individual. Acid-activated proteolysis of IGFBP-1 was completely removed by heparin affinity chromatography (FIG. 6B,C).

The protease activity in this urine was inhibited by PMSF (10 mM), chumostatin (10 μM), and the kallikrein inhibitor cyclohexylacetyl-Phe-Arg-Ser-Val-Gln (100 μM) (FIG. 7A). It was notable that aprotinin, which can bind azurocidin was either without effect, or even promoted protease activity in different experiments. Not shown here, TPCK (1 mM) inhibited proteolysis, while TLCK did not. Proteolysis was inhibited by divalent metal cations, such as CuCl₂, NiCl₂, and CoCl₂, while CaCl₂ had a significant promoting effect on activity (FIG. 7B). EDTA, EGTA and 1,10-phenothroline had no effect on the degree of IGFBP-1 degradation (data not shown). Partial inhibition of IGFBP-1 proteolysis was also seen in the presence of IGF-I and IGF-II, with no effect of insulin (FIG. 8A) or des(1-3)-IGF-I (FIG. 8B). There was no difference in the degree of proteolysis when protease was pre-incubated with IGFs or insulin and then incubated with IGFBP-1, compared to preincubation of IGFs with IGFBP-1 and then exposure to the protease (data not shown).

The time course of degradation of phosphorylated and non-phosphorylated IGFBP-1 was also compared (FIG. 9) and no difference was found in the rate of degradation of each isoform, with fragments visible within 3 min, and substantial degradation by 2 h under these conditions.

The immunoreactivity of proteolysed IGFBP-1 was characterised. In these experiments native pIGFBP-1 was used. Concentrations were measured by radioimmunoassay using two polyclonal antibodies raised against recombinant pIGFBP-1 (SU12) and amniotic fluid-purified IGFBP-1 (A2). In FIG. 10A we show that there was no obvious effect of proteolysis on the displacement curves for HepG2-purified (highly phosphorylated) IGFBP-1 or the dephosphorylated isoform in the radioimmunoassays. A decrease in intact IGFBP-1 and an increase in IGFBP-1 immunoreactivity at 14 kDa after proteolysis were seen on immunoblotting (FIG. 10B). No fragments of IGFBP-1 after azurocidin-dependent proteolysis were detectable on ligand binding after SDS-PAGE (FIG. 10B).

When biotinylated IGFBP-1 is used as substrate, N-terminal fragments migrating at 14, 20 and 22 kDa are seen after proteolysis (FIG. 10C). This contrasts with radioiodinated npIGFBP-1 (FIGS. 6-9) or pIGFBP-1 (FIG. 9) are used; or when pIGFBP-1 fragments are detected by immunoblotting (FIG. 10B) where proteolysis results in forms of approx. 14 kDa on SDS-PAGE.

When unlabelled IGFBP-1 is cleaved and detected by silver staining after SDS-PAGE, the major fragments are 14 and 20 kDa (FIG. 11A, lane 1). When these bands were excised and the tryptic digests analyzed by MALDI-MS, both mol mass bands contained sequences corresponding to the N- and C-terminal regions of IGFBP-1 (FIG. 11B).

Proteolysed npIGFBP-1 was subjected to HPLC and purified fragments were also visualized on SDS-PAGE (FIG. 6A, lanes 3-9). Those corresponding to the N-terminal region of IGFBP-1 migrated at 20 kDa, while fragments corresponding to the C-terminal region migrated at 14 kDa. Similar results were obtained for recombinant pIGFBP-1 (data not shown). N-terminal sequence analysis was performed on 4 of the HPLC runs, two using recombinant npIGFBP-1 and two using recombinant pIGFBP-1. These results are summarized in FIG. 11B. The primary cleavage site, present in all 4 runs, is at Ile¹³⁰-Ser¹³¹ as reported above. This was confirmed by C-terminal sequence analysis on one of the runs. A secondary cleavage site, present in 3 of the HPLC runs, was at Val¹⁴¹-Thr¹⁴². Three other cleavage sites, observed in only one run each, occurred after isoleucine at Ile¹⁵³-Glu¹⁵⁴ and Ile¹⁷³-Ser¹⁷⁴; and after valine at Val¹⁵⁹-Glu¹⁶⁰. For three of the HPLC runs IGFBP-1 was degraded with the partially purified azurocidin. In one run urine was used and further cleavage site at Thr¹³²-Tyr¹³³ was seen, which probably represents cleavage by the pepsin, or a pepsin-like protease in this material.

The interaction of proteolysed IGFBP-1 with IGF-I and IGF-II biosensor surfaces was analysed (BIAcore). In FIG. 12 the mean biosensorgrams for the interaction of 200 nM phosphorylated IGFBP-1 intact are shown, and after 2-h and 4-h incubations with partially purified azurocidin, with immobilized IGF-I (FIG. 12A) and IGF-II (FIG. 12B). The kinetic analyses of the rates of association (over 21-120 s, ks) and dissociation (over 261-360 s, kd) for the results shown in FIGS. 12A and 12B are summarized in Table 2. Proteolysis of 200 mM IGFBP-1 for 2 h resulted in a greater than 3-fold increase in the rates of association and dissociation when analyzed across both IGF-I and IGF-II. The rate of dissociation further increased to more than 7-fold at the 4-h time point. The rate of dissociation of IGFBP-1 from IGF-II was greater than twice that from IGF-I, and proteolysis had no effect on this relationship. The apparent ka was calculated from the slope of the association rate (ks) and various concentrations of IGFBP-1 (FIGS. 12C and 12D), and was 1.87±0.34×104 M-1s-1 for the interaction between IGFBP-1 and IGF-1 and 1.68±0.38×104 M-1 s-1 for the interaction between IGFBP-1 and IGF-II. The apparent ka increased after proteolysis to 8.30±0.10×104 M-1 s-1 for the interaction between 2-h proteolysed IGFBP-1 and IGF-I and 8.01±0.35×104 M-1s-1 for IGF-II. However there was also an increase in the maximum biosensor response for the IGF-I chip, suggesting an increase in the number of IGF-binding sites after proteolysis. At the 2-h time point of proteolysis the predicted maximum response for interaction with IGF-I, based on the initial rate of association (21-120 s), was 1156±16 RU, significantly greater than 908±79 RU for intact IGFBP-1 (P=0.011).

The interaction of IGFBP-1 with des(1-3)IGF-I on the biosensor surface was also determined. In FIG. 13 we show the mean biosensorgrams for the interaction of 200 nM phosphorylated IGFBP-1, intact and after 2-h and 4-h incubations with partially purified azurocidin. The biosensor response for the interaction between IGFBP-1 and des(1-3)IGF-I was 10-fold less than with full length IGF-I. Proteolysis resulted in a clearly increase in the rate of dissociation. Detailed analysis of ks and kd of the des(1-3)IGF-I curves was not possible.

TABLE 2
Kinetic analysis of interactions of intact and proteolysed
phosphorylated IGFBP-1 with IGF biosensor surfaces
The kinetic data shown in FIG. 6 were analysed using the 1:1 Langmuir interaction model.
IGFBP-I was injected either intact, or after 2- or 4-proteolysis by azurocidin, over IGF-I
and IGF-II biosensor surfaces. The rates of association (over 21-120 s, kₛ) and
dissociation (over 261-360 s, k_{d}) are expressed as the mean ± SEM. The rates relative to
that of 200 nM intact pIGFBP-1 and to that of IGF-I are also shown.

| | IGFBP-1 | proteolysis | kₛ | | rel to | k_{d} | | rel to |
|---|---|---|---|---|---|---|---|---|
| | nM | h | ×10⁻³ s⁻¹ | rel kₛ | IGF-I | ×10⁻⁴ s⁻¹ | rel k_{d} | IGF-I |
| IGF-I | 400 | 0 | 8.21 ± 0.14 | 1.91 | | 1.45 ± 0.02 | 1.00 | |
| | 200 | 0 | 4.29 ± 0.46 | 1.00 | | 1.46 ± 0.19 | 1.00 | |
| | 200 | 2 | 17.26 ± 0.49 | 4.02* | | 6.40 ± 0.25 | 4.40* | |
| | 200 | 4 | 12.69 ± 0.08 | 2.96*^{†} | | 11.36 ± 0.05 | 7.81*^{†} | |
| IGF-II | 400 | 0 | 8.30 ± 0.13 | 1.86 | 1.01 | 3.37 ± 0.02 | 0.97 | 2.33^{‡} |
| | 200 | 0 | 4.47 ± 0.78 | 1.00 | 1.02 | 3.48 ± 0.40 | 1.00 | 2.41^{‡} |
| | 200 | 2 | 15.43 ± 0.58 | 3.45* | 0.89 | 15.99 ± 0.19 | 4.59* | 2.51^{‡} |
| | 200 | 4 | 12.80 ± 0.08 | 2.86*^{†} | 1.01 | 27.17 ± 0.12 | 7.81*^{†} | 2.39^{‡} |

*P < 0.001, relative to 200 nM intact IGFBP-1
^{†}P < 0.001, relative to 200 nM IGFBP-1, proteolysed 2 h
^{‡}P < 0.001, relative to IGF-I

### Example 3

### Biological Effects of Intact and Proteolysed IGFBP-1

### Methods

Cell studies. MCF-7 human breast cancer cells were maintained in MEM alpha ribonucleic acid-free medium supplemented with 10% foetal bovine serum, 50 Unit/ml penicillin sodium, 50 μg/ml streptomycin sulfate and 2 mM L-glutamine at 37C in 5% CO₂ humidified environment.

Human skeletal muscle cells were isolated, with approval from the local Ethics Committee, from the muscle biopsies by trypsin digestion and were grown to confluent myoblasts and differentiated into myotubes as previously described in detail (Bouzakri et al, Diabetes, 52, 1319-1325, 2003). Muscle from 4 different subjects were individually minced before being incubated for 30 min in 5 ml of trypsin-EDTA (Invitrogen, Carlsbad, Calif.) at 37° C. under agitation. After being centrifuged (150 g), the pellet was rinsed several times in PBS. Cells were first preplated for 1 h to eliminate rapidly adherent fibroblasts. The remaining cells were then cultured in a growth medium composed of HAM F-10 supplemented with 20% fetal bovine serum (FBS; Invitrogen Stockholm, Sweden), and 1% antibiotics (Invitrogen Stockholm, Sweden). Cells were plated in 6 wells plates and cultured in growth medium at 37° C. until confluence. Differentiation into myotubes was induced by changing the medium to Dulbecco's modified Eagle's medium supplemented with 2% FBS, and 1% antibiotics. Cells showed polynucleated status and expressed specific markers of human skeletal muscle 4 days after differentiation was initiated (20).

3H-Thymidine incorporation: Experiments were carried out in 48-well plates, at 70% confluence. After a 20-h incubation in serum-free medium supplemented with 0.1% BSA, the experimental conditions were added for a further 24 h, with ³H-thymidine (1 μCi/ml) added for the last 4 h. Cells were then washed with ice-cold 0.9% NaCl three times and incubated with ice-cold 5% TCA for 15 min at 22 C. The fixed nucleotides were solubilized in 0.1M NaOH at 22 C and subjected to liquid scintillation counting.

2-Deoxyglucose transport: Glucose uptake was performed as previously described for primary human muscle cells (Bouzakri et al, Diabetologia, 47, 1314-1323, 2004). After 24-h serum starvation, myotubes were pre-incubated with or without IGF-II (10 ng/ml) and IGF-II+IGFBP-1 (intact or cleaved at 20 or 80 ng/ml) for 90 min. IGF-II and IGFBP-1 were mixed 30 min before the experiments. Cells were then washed four times with pre-warmed buffer (150 mmol/l NaCl, 5 μmmol/l KCl, 1.2 mmol/l MgSO₄, 1.2 mmol/l CaCL₂, 2.5 mmol/l NaH₂PO₄, 10 mmol/l HEPES, pH 7.4) and glucose uptake was measured for 15 min using [3H]2-deoxyglucose (50 μmol/l, 14,800 Bq/well). Results were expressed as the percentage of glucose uptake in the absence of IGF-II or IGFBP-1.

Statistical analyses. The effect of IGF-II and IGFBP-1 in muscle cell culture was analyzed by two-way ANOVA. Where significant differences were observed, comparisons were made using the Tukey test. In the study using MCF-7 breast cancer cells the Wolcoxon Matched Pairs test was used. Statistical significance was set at P<0.05.

### Results

Biological effects of intact and proteolysed IGFBP-1. The effect of pIGFBP-1 on IGF-II action was studied in cultured cells. In the human breast cancer cell line, MCF-7, IGF-II 10 ng/ml stimulated 3H-thymidine incorporation 2-fold (range 1.5-2.7; 6 experiments, P=0.028). Addition of intact pIGFBP-1 90 ng/ml decreased this to 1.5-fold (range 1.2-2.1; 6 experiments, P=0.028), whereas no inhibition was seen when proteolysed IGFBP-1 was added. In 4 of the experiments the effect of pIGFBP-1 alone was also determined, and resulted in a significant inhibition of 3H-thymidine incorporation with intact pIGFBP-1 (range 13-33%, P=0.030), and not with proteolysed pIGFBP-1. The effect of pIGFBP-1 on glucose uptake was determined in primary human skeletal myoblasts after differentiation into myotubes (FIG. 15). A submaximal concentration of IGF-II, 10 ng/ml, stimulated glucose uptake more than 2-fold (P<0.001) and was inhibited in a dose-response manner, by both intact and proteolysed IGFBP-1. The inhibitory effect of the proteolytic fragments of IGFBP-1 was significantly less that that of intact IGFBP-1 (P<0.001). Proteolysed IGFBP-1, in the absence of exogenous IGF-II, stimulated glucose uptake (80 ng/ml; P<0.001), while intact pIGFBP-1 had no effect alone.

### Discussion

A protease that is specific for IGFBP-1 has been discovered, which efficiently cleaves both the non-phosphorylated isoform and the phosphorylated form, with its high affinity for IGF-I. Cleavage of pIGFBP-1 by this protease generated fragments that together have higher association and dissociation rates for IGFs compared to the intact protein. It also demonstrated a higher total capacity for IGF-I binding, suggesting that both and N- and C-terminal fragments may interact with ligand independently of each other. This would result in an increase in IGF turnover and availability to tissues. In human MCF-7 breast cancer cells and in human primary skeletal muscle cells, it has been shown that the inhibitory effect of pIGFBP-1 on IGF-II-stimulated DNA synthesis and glucose uptake is decreased after proteolysis.

IGFBP-1 is cleaved at a unique site by this novel protease activity. The P1 specificity was for isoleucine, and also valine, both of which are hydrophobic amino acids. The specificity of the cleavage site is consistent with predications for azurocidin activity, based on phage display selection of P1 mutants of aprotinin. The primary site of cleavage was at Ile¹³⁰-Ser¹³¹, in a region that is highly conserved in IGFBP-1 between species, and is not found in other IGFBPs. The serine residue at the site has not been reported as significantly phosphorylated, whereas Ser¹⁰¹, Ser¹¹⁹, ser¹⁶⁹ are sites of phosphorylation that increase the affinity for IGF-I, and confer resistance to previously described proteases. Cleavage generated a 130-amino acid N-terminal fragment that, when purified, migrated at 20 kDa on SDS-PAGE; and a 104-amino acid C-terminal fragment. Further cleavage at Val¹⁴¹-Thr¹⁴² was often seen, generating a 93-amino acid fragment that, when purified, migrated at 14 kDa. When IGFBP-1 is cleaved and subjected to SDS-PAGE, and tryptic digests of the 20 and 14 kDa bands analyzed, sequences corresponding to both N- and C-terminal regions are detected. The C-terminal fragment may form dimers and that there is some further degradation of the α-terminal fragment. This is supported by the observation that 20- and 14-kDa are visible after cleaving IGFBP-1 that is biotinylated on the N-terminus. Both of the C-terminal fragments that have been identified (IGFBP-1¹³¹⁻²³⁴ and IGFBP-1¹⁴²⁻²³⁴) contain the RGD sequence of IGFBP-1 and are therefore expected to retain activity through interaction with the α₅β₁ integrin receptor. A C-terminal fragment isolated from amniotic fluid starting at Val¹⁴¹, is reported to have an IGF-independent effect on cell migration compared to intact IGFBP-1. Whether the effect on glucose uptake into human skeletal muscle cells by proteolysed IGFBP-1 alone could be attributed to an IGF-independent effect via the RGD sequence has to be elucidated in future studies.

Azurocidin is the specific IGFBP-1 protease based on (i) its presence in the partially purified active material, (ii) the inhibitory effect of a monoclonal antibody to azurocidin (iii) immunodepletion of active protease using this antibody and (iv) clear differences in the pattern of fragmentation and stability of fragments compared to other candidate proteases. The recombinant azurocidin that is commercially available lacked activity on IGFBP-1 and had a lower mol mass on SDS-PAGE, which is likely to be due to differences in N-glycosylation. This glycosylation difference, or another post-translational difference likely accounts for the lack of protease activity. Alternatively, activity may have been lost during purification.

The fact that azurocidin may be a specific protease was unexpected.

Although closely structurally related to three other neutrophil granule serprocidins, elastase, proteinase 3 and cathepsin G, azurocidin is generally regarded as proteolytically inactive due to loss of the catalytically active Ser¹⁷⁵. However human azurocidin has fisher residue differences at the active site versus human elastase and proteinase 3, and has by additional replacements gained Ser⁴¹ as well as other residues towards other serprocidins, such that adjacent Ser and Asp residues still exist at the active site pocket, possibly supplying the necessary residues for catalytic proteolysis. Elastase and cathepsin G have been described to cleave IGFBP-1, but also degrade all of the IGPBPs, with IGFBP-5 and IGFBP-3 and -4, respectively as preferred substrates. It has been shown that proteinase 3 can also cleave IGFBP-1. However, for each of these three proteases, there were clear differences in the patterns of fragmentation, and in the stability of the IGFBP-1 fragments compared to those generated by our protease. The stability of the IGFBP-1 fragments generated in the presence of azurocidin was remarkable, and contrasts to the effect of other proteases, with no further degradation seen after 24-h incubation at 37 C or after a 2-h incubation at 45 C.

Azurocidin is a multifunctional protein which is readily mobilized from secretory granules and is antimicrobial, chemotactic and induces cytokine secretion. It therefore has an important roles in host defence in inflammation and infection. The proteolytic effect on IGFBP-1 is noticeable at a nanomolar concentration of azurocidin in the purified preparation, substantially less than that required for other effects of azurocidin. For example monocyte chemotaxis requires a micromolar concentration. The optimal pH also differs. Its bactericidal action is maximal in the acidic conditions that are present in lysosomal granules, and this activity decreases by at least 50% when the pH is increased from 5.5 to 6.5. Its action on IGFBP-1, however, is greater above pH 6.

This IGFBP-1 protease was isolated from the urine of an individual with multiple myeloma and atopy, and succeeded in separating it from an acid-activated pepsin-like protease. The tissue source of the IGFBP-1 protease activity in this urine is not known, however it was co-purified with myeloperoxidase and lactoferrin, proteins that are also abundant in polymorphonuclear leukocytes. The pathophysiological significance of IGFBP-1-specific proteolysis in an individual with multiple myeloma is yet to be clarified. To our knowledge expression of azurocidin has not been reported in any lymphoid lineage and it seems likely that inflammatory cells (neutrophils), which are associated with this malignancy, are the source. It appears possible that the presence of active azurocidin through IGFBP-1 proteolysis might increase myeloma cell growth and survival. In addition to its expression in neutrophils, azurocidin is expressed in vascular endothelial cells in response to inflammatory mediators. Its expression is associated, for example, with atherosclerotic lesions.

IGFBP-1 and azurocidin both have important roles in inflammation and catabolism. IGFBP-1 increases dramatically in catabolic states, circulates in the phosphorylated isoform, accumulates in peripheral tissues, including skeletal muscle and inhibits IGF-I action. Our finding that it can be degraded is therefore of considerable physiological and pharmacological interest. The generation of IGF-binding fragments with lower IGF affinity will increase turnover and IGF availability in target tissues. Hence a new factor in the regulation of IGF activity has been identified.

1-32. (canceled)

33. A method of treating renal disease, growth impairment in children with diabetes or a condition associated with hepatic insulin resistance, said method comprising administering azurocidin to a subject in need thereof.

34. The method of claim 33, wherein said condition associated with hepatic insulin resistance is associated with a reduced ability to suppress hepatic glucose output, VLDL triglyceride production and/or IGFBP-1 production.

35. The method of claim 33, wherein said condition associated with hepatic insulin resistance comprises elevated serum IGFBP-1 levels.

36. The method of claim 33, wherein said condition associated with hepatic insulin resistance is selected from any one or more of critical illness with accompanying hepatic insulin resistance, intensive care with the accompanying hepatic insulin resistance, type II diabetes, impaired glucose tolerance and type I diabetes associated with hepatic insulin resistance.

37. The method of claim 33, wherein the azurocidin is administered as a dose which results in tissue levels of azurocidin of 1-10 nmol/L.

38. A method of treating a condition associated with IGFBP-1 and/or IGF level and/or activity, wherein said method of comprises administering a modulator which blocks or enhances the interaction between azurocidin and IGFBP-1 to a subject in need thereof.

39. The method of claim 38, wherein said modulator is an antibody, a peptide containing the specific cleavage site in IGFBP-1 for azurocidin or aprotinin.

40. The method of claim 39, wherein said antibody is an anti-azurocidin antibody, or an anti-IGFBP-1 antibody.

41. The method of claim 38, wherein said modulator is a peptide of 4 to 20 amino acids comprising an Ile residue where said peptide is capable of acting as substrate for azurocidin protease activity.

42. The method of claim 41, wherein said peptide is of formula I
(X)n-I—(X)m
wherein X is any amino acid; n is 1 to 10; and m is 0 to 10; and wherein n and m may be the same or different.

43. The method of claim 41, wherein said peptide is the octapeptide WDAISTYD or a fragment thereof which retains I and is capable of acting as a proteolytic substrate for azurocidin.

44. The method of claim 38, wherein said condition is associated elevated levels of IGFBP-1 or is associated with an unwanted level or activity of IGF.

45. The method of claim 44, wherein said condition is selected from septic shock, trauma, critical illness, intensive care, a condition associated with hepatic insulin resistance, renal disease or growth impairment in children with diabetes or wherein said condition is cancer or a condition associated with inflammation.

46. A peptide comprising the amino acid sequence WDAISTYD or a fragment thereof which retains I, wherein said peptide or fragment thereof is capable of acting as a proteolytic substrate for azurocidin.

47. A pharmaceutical composition comprising azurocidin and IGFBP-1 or an anti-azurocidin antibody, together with at least one pharmaceutically acceptable carrier, excipient or diluent.

48. A product containing azurocidin and IGFBP-1 as a combined preparation for simultaneous, separate or sequential use in would-healing.


SEQUENCE LISTING (23 sequences)

SEQ ID NO: 1 - protein, 225 aa, Homo sapiens
IVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 2 - protein, 227 aa, Homo sapiens
LDIVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 3 - protein, 232 aa, Homo sapiens
GSSPLLDIVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 4 - protein, 251 aa, Homo sapiens
MTRLTVLALLAGLLASSRAGSSPLLDIVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 5 - DNA, 805 bp, Homo sapiens
tagccacagacctgccccgccatgacccggctgacagtcctggccctgctggctggtctgctggcgtcctcgagggccggctccagcccccttttggacatcgttggcggccggaaggcgaggccccgccagttcccgttcctggcctccattcagaatcaaggcaggcacttctgcgggggtgccctgatccatgcccgcttcgtgatgaccgcggccagctgcttccaaagccagaaccccggggttagcaccgtggtgctgggtgcctatgacctgaggcggcgggagaggcagtcccgccagacgttttccatcagcagcatgagcgagaatggctacgacccccagcagaacctgaacgacctgatgctgcttcagctggaccgtgaggccaacctcaccagcagcgtgacgatactgccactgcctctgcagaacgccacggtggaagccggcaccagatgccaggtggccggctgggggagccagcgcagtggggggcgtctctcccgttttcccaggtttgtcaacgtgactgtgacccccgaggaccagtgtcgccccaacaacgtgtgcaccggtgtgctcacccgccgcggtggcatctgcaatggggacgggggcacccccctcgtctgcgagggcctggcccacggcgtggcctccttttccctggggccctgtggccgaggccctgacttcttcacccgagtggcgctcttccgagactggatcgatggtgttctcaacaacccgggaccggggccagcctaggggggcctgtgacctcccatggagccca

SEQ ID NO: 6 - protein, 100 aa, Homo sapiens
MKMSRLCLSVALLVLLGTLAASTPGCDTSNQAKAQRPDFCLEPPYTGPCKARIIRYFYNAKAGLCQTFVYGGCRAKRNNFKSAEDCMRTCGGAIGPWENL

SEQ ID NO: 7 - DNA, 302 bp, Homo sapiens
atgaagatgagccggctctgcctctccgtagcccttctggtcctcctgggcaccctggcggccagcactccagggtgtgacaccagcaaccaggccaaagcccagcggcctgacttctgcctagagcctccatatacgggtccctgcaaggccagaattatcagatacttctacaacgccaaggctgggctctgccagacctttgtatatggcggctgcagagctaaaagaaacaattcaagagcgcagaggactgcatgaggacctgtggtggtgctattgggccctgggagaacctgtga

SEQ ID NO: 8 - protein, 234 aa, Artificial Sequence
  Synthetic
APWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 9 - protein, 21 aa, Artificial Sequence
  Synthetic
XXXXXXXXXXIXXXXXXXXXX

SEQ ID NO: 10 - protein, 8 aa, Artificial Sequence
  Synthetic
WDAISTYD

SEQ ID NO: 11 - protein, 4 aa, Artificial Sequence
  Synthetic
WDAI

SEQ ID NO: 12 - protein, 3 aa, Artificial Sequence
  Synthetic
DAI

SEQ ID NO: 13 - protein, 4 aa, Artificial Sequence
  Synthetic
DAIS

SEQ ID NO: 14 - protein, 5 aa, Artificial Sequence
  Synthetic
WDAIS

SEQ ID NO: 15 - protein, 5 aa, Artificial Sequence
  Synthetic
DAIST

SEQ ID NO: 16 - protein, 6 aa, Artificial Sequence
  Synthetic
WDAIST

SEQ ID NO: 17 - protein, 6 aa, Artificial Sequence
  Synthetic
DAISTY

SEQ ID NO: 18 - protein, 7 aa, Artificial Sequence
  Synthetic
WDAISTY

SEQ ID NO: 19 - protein, 7 aa, Artificial Sequence
  Synthetic
DAISTYD

SEQ ID NO: 20 - protein, 3 aa, Artificial Sequence
  Synthetic
AIS

SEQ ID NO: 21 - protein, 4 aa, Artificial Sequence
  Synthetic
AIST

SEQ ID NO: 22 - protein, 5 aa, Artificial Sequence
  Synthetic
AISTY

SEQ ID NO: 23 - protein, 6 aa, Artificial Sequence
  Synthetic
AISTYD